(19) United States
(12) Patent Application Publication
(10) Pub. No.: US 2026/0088132 A1
(43) Pub. Date: Mar. 26, 2026

(54) CHEMICAL PAINTING

(71) Applicant: The Trustees of Columbia University in the City of New York, New York, NY (US)

(72) Inventors: Xinwen LIU, New York, NY (US); Wei MIN, New York, NY (US)

(21) Appl. No.: 19/404,733

(22) Filed: Dec. 1, 2025

Related U.S. Application Data
(63) Continuation of application No. PCT/US2024/031251, filed on May 28, 2024.
(60) Provisional application No. 63/470,509, filed on Jun. 2, 2023.
(60) Provisional application No. 63/527,990, filed on Jul. 20, 2023.

(51) Int. Cl. G16B 40/10; G01N 21/35; G01N 21/65; G01N 33/50; G16B 25/10

(52) U.S. Cl. CPC G16B 40/10; G01N 21/35; G01N 21/65; G01N 33/5008; G16B 25/10

(57) ABSTRACT

Systems, methods and devices for profiling phenotypes of cells, and associated methods for identifying or predicting drug mechanism of action, discovering drug candidates with novel mechanisms of actions, differential subpopulation responses to drugs, and novel IR vibrational probes.

### CROSS-REFERENCE TO RELATED APPLICATIONS

This application is a continuation of PCT International Application No. PCT/US2024/031251, filed May 28, 2024, which claims benefit of U.S. Provisional Application No. 63/470,509, filed Jun. 2, 2023, and U.S. Provisional Application No. 63/527,990, filed Jul. 20, 2023, the contents of each of which are hereby incorporated by reference.

### STATEMENT OF GOVERNMENTAL INTEREST

This invention was made with government support under grant number EB029523 awarded by the National Institutes of Health. The government has certain rights in the invention.

### BACKGROUND

The disclosures of all publications, patents, patent application publications and books referred to in this application are hereby incorporated by reference in their entirety into the subject application to more fully describe the art to which the subject invention pertains.

Cellular drug responses refer to the changes that occur in cells in response to drugs, including alterations in gene expressions, protein abundance, metabolism, etc. [1]. The study of cellular drug responses is vital to drug discovery, as it facilitates understanding of drug mechanism of action (MoA), evaluating drug efficacy, overcoming drug resistance, and optimizing drug therapy [2,3]. However, drug response in many diseases varies dramatically due to the complex nature of cellular phenotypes and disease context [4]. Even among the same kind of cells in the same human tissue, there exists obvious heterogeneity in molecular phenotypes and gene expression [5]. Consequently, the average result from traditional ensemble measurement might deviate significantly from actual cellular drug responses and mask cell-to-cell heterogeneity. Therefore, it has been suggested that only the drug response of a single-cell can more accurately reflect drug efficacy [6].

Despite the perceived importance of single-cell drug response in drug discovery, its measurements present significant challenges. An ideal technique should satisfy the following requirements [5]: (1) sufficient signal-to-noise ratio (SNR) on single-cell; (2) high throughput to measure a large number of cells; (3) high content to detect multiple cell features; (4) non-invasive to measure drug responses from intact or live cells; (5) low device cost and ease-of-operation to adapt to large-scale drug research; (6) robust performance with minimal batch effects. Current techniques for single-cell drug response can be mainly divided into three categories, including optical methods, mass spectrometry methods and single-cell sequencing methods. Unfortunately, none of them can fully satisfy the mentioned criteria. For optical methods, label-free fluorescence metabolic imaging measures the signal from autofluorescent metabolic coenzymes (reduced NADH and FAD) [7] but is constrained by its low content. Image-based profiling methods such as Cell Painting [8] provide enriched morphological content from organelle staining. However, they are limited by batch effects and plate layout effects for identifying drug MoAs [3,9]. Mass spectrometry methods can offer multiplexed metabolic or antigenic cell features at single-cell level, but they are intrinsically destructive and require expensive instruments [10-14]. Single-cell RNA sequencing methods provide valuable insights into the drug-induced molecular changes [15-17]. Nevertheless, they still suffer from high costs and complicated operations for large-scale drug development.

### BRIEF SUMMARY OF THE INVENTION

A method of profiling a phenotype of a cell comprising:
introducing a plurality of different IR vibrational probes or Raman vibrational probes into the cell, wherein the plurality of different vibrational probes comprise at least two different probes which each emit a quantifiable signal corresponding to a unique cellular parameter; and
obtaining and/or quantifying, by vibrational probe spectroscopy or by vibrational probe imaging, a signal of each vibrational probe so as to determine and/or quantify presence, abundance, and/or spatial location of at least two unique cellular parameters in the cell, so as to thereby profile the phenotype of the cell.

A method of profiling a phenotype of a cell comprising:
introducing at least one IR vibrational probe or Raman vibrational probe into the cell, the at least one probe which can emit a quantifiable signal corresponding to a unique cellular parameter; and
obtaining and/or quantifying, by vibrational probe spectroscopy or by vibrational probe imaging, a signal of the at least one vibrational probe so as to determine and/or quantify presence, abundance, and/or spatial location of at least one unique cellular parameter in the cell, so as to thereby profile the phenotype of the cell.

A method of profiling a phenotype of a cell is provided comprising:
introducing a plurality of different infrared (IR) vibrational probes or Raman vibrational probes into the cell, wherein the plurality of different vibrational probes comprise at least two different probes which each emit a quantifiable signal in response to a different cellular parameter; and
obtaining and/or quantifying, by vibrational probe spectroscopy or by vibrational probe imaging, a signal of each vibrational probe so as to determine and/or quantify presence, abundance and/or spatial location of two or more cellular parameters in the cell, so as to thereby profile the phenotype of the cell.

A method is provided for classifying a candidate drug, or a combination of candidate drugs, as having a class of biological mechanism of action at a single cell level comprising:
obtaining a quantified single cell test imaging signal or test spectroscopy signal generated by a plurality of IR vibrational probes and quantified from a cell treated with the candidate drug or combination, the cell comprising therein a plurality of IR vibrational probes comprising at least two of the following probes: a probe for protein synthesis; a probe for lipid metabolism, lipid synthesis or lipid uptake; a probe for carbohydrate synthesis; and a probe for nucleic acid intercalation;
providing the quantified single cell test imaging signal or test spectroscopy signal as an input to a machine learning classifier module utilizing a linear discriminant analysis algorithm and a training set comprising a plurality of quantified single cell imaging signals or spectroscopy signals labeled as to class of biological mechanism of action and quantified from a plurality of cells containing IR vibrational probes wherein the cells have been treated with a drug having a known class of biological mechanism of action, the IR vibrational probes comprising at least two of the following probes: a probe for protein synthesis; a probe for lipid metabolism, lipid synthesis or lipid uptake; a probe for carbohydrate synthesis; and a probe for nucleic acid intercalation, such that the machine learning classifier module determines as an output the class of biological mechanism of action of the candidate drug, or of the combination of candidate drugs; and
transmitting the class of biological mechanism of action of the candidate drug or candidate drug combination from the machine learning classifier module to a user device and/or user display.

A method is provided for classifying a candidate drug, or a combination of candidate drugs, as having a class of biological mechanism of action at a single cell level comprising:
obtaining a quantified single cell test imaging signal or test spectroscopy signal generated by an IR vibrational probe and quantified from a cell treated with the candidate drug or combination, the cell comprising therein an IR vibrational probe comprising at least one of the following probes: a probe for protein synthesis; a probe for lipid metabolism, lipid synthesis or lipid uptake; a probe for carbohydrate synthesis; and a probe for nucleic acid intercalation;
providing the quantified single cell test imaging signal or test spectroscopy signal as an input to a machine learning classifier module utilizing a linear discriminant analysis algorithm and a training set comprising a plurality of quantified single cell imaging signals or spectroscopy signals labeled as to class of biological mechanism of action and quantified from a plurality of cells containing IR vibrational probes wherein the cells have been treated with a drug having a known class of biological mechanism of action, said IR vibrational probe comprising at least one of the following probes: a probe for protein synthesis; a probe for lipid metabolism, lipid synthesis or lipid uptake; a probe for carbohydrate synthesis; and a probe for nucleic acid intercalation, such that the machine learning classifier module determines as an output the class of biological mechanism of action of the candidate drug, or of the combination of candidate drugs; and
transmitting the class of biological mechanism of action of the candidate drug or candidate drug combination from the machine learning classifier module to a user device and/or user display.

A method is provided for method of classifying a candidate drug, or a combination of candidate drugs, as having a class of biological mechanism of action at a single cell level comprising:
obtaining a quantified single cell test imaging signal or test spectroscopy signal generated by a cell comprising a plurality of IR vibrational probes or Raman vibrational probes therein, wherein the plurality of different vibrational probes comprise at least two different probes which each emit a quantifiable signal in response to a different cellular parameter, wherein the cell has been treated with the candidate drug or combination;
providing the quantified single cell test imaging signal or test spectroscopy signal as an input to a machine learning classifier module utilizing a linear discriminant analysis algorithm and a training set comprising a plurality of quantified single cell imaging signals or spectroscopy signals labeled as to class of biological mechanism of action and quantified from a plurality of cells containing IR vibrational probes wherein the cells have been treated with a drug having a known class of biological mechanism of action, wherein the plurality of different vibrational probes comprise at least two different probes which each emit a quantifiable signal in response to a different cellular parameter, such that the machine learning classifier module determines as an output the class of biological mechanism of action of the candidate drug, or of the combination of candidate drugs; and
transmitting the class of biological mechanism of action from the machine learning classifier module to a user device and/or user display.

A method is provided for identifying a novel biological mechanism of action for an unannotated drug candidate, or for identifying a biological mechanism of action of an unannotated drug combination, not previously known to have said biological mechanism of action comprising: providing a phenotype profile of a cell treated with said unannotated drug, or said unannotated drug combination, which phenotype profile has been obtained by the methods described herein, comparing the phenotype profile with one or more reference classes in a model obtained by applying linear discriminant analysis as a machine learning classifier to a training set of quantified single cell data, quantified from a plurality of single cells, of signals a plurality of vibrational probes comprising at least two of the following probes: a probe for protein synthesis; a probe for lipid metabolism, lipid synthesis or lipid uptake; a probe for carbohydrate synthesis; and a probe for nucleic acid intercalation, so as to provide a prediction model of a biological mechanism of action of a candidate drug, or of combination of candidate drugs, for one or more reference classes comprising protein synthesis mechanism of action; lipid metabolism mechanism of action, lipid synthesis mechanism of action, lipid uptake mechanism of action; carbohydrate synthesis mechanism of action; nucleic acid intercalation mechanism of action;
determining a Mahalanobis distance between the phenotype profile of a cell treated with said unannotated drug, or said unannotated drug combination, with the one or more reference classes in the model so as to determine the reference class with a smallest Mahalanobis distance from the phenotype profile of the so-treated cell;
providing a database of drugs and/or combination of drugs known to have the biological mechanism of action of said reference class with the smallest distance from the phenotype profile of the so-treated cell; and
applying an isolation forest so as to determine whether the unannotated drug or unannotated drug combination having said biological mechanism of action of said reference class.

A method is provided for identifying differential phenotype responses to a drug among a plurality of cells from a sample comprising performing the method described herein on a sample comprising a plurality of cells, which sample has been subjected to said drug, so as to determine whether the phenotype responses to a drug among the cells of the sample are homogenous or differ, wherein non-homogeneous phenotype responses to the drug among the cells identify a differential phenotype response to the drug among the plurality of cells.

A device is provided comprising a vibrational infrared (IR) detection device comprising a sample chamber, which vibrational IR detection device comprises a radiation source which irradiates the sample chamber with radiation of mid-infrared wavelengths (1000-3500±200 cm⁻¹) and a detector which detects and quantifies absorption spectra of a plurality of mid-infrared probes corresponding to one or more predetermined metabolic processes by a cell within a sample within said sample chamber so as to obtain spatial and quantitative data about said predetermined metabolic processes by a cell, and wherein said device presents to a user thereof the spatial and/or quantitative data from the plurality of mid-infrared probes.

A system is provided comprising a device described herein, a biological sample in a sample chamber thereof, and a plurality of mid-infrared probes within the biological sample corresponding to one or more predetermined cellular metabolic processes.

A system is provided comprising a device described herein, comprising an irradiation source, a beam splitter, a moving mirror, and a fixed mirror.

A system is provided comprising:
one or more computers operatively connected to memory wherein the memory stores computer readable instructions comprising program code that, when executed, cause the one or more computers to perform the steps of:
obtaining a quantified single cell test imaging signal or test spectroscopy signal generated by a plurality of IR vibrational probes and quantified from a cell treated with the candidate drug or combination, the cell comprising therein a plurality of IR vibrational probes comprising at least two of the following probes: a probe for protein synthesis; a probe for lipid metabolism, lipid synthesis or lipid uptake; a probe for carbohydrate synthesis; and a probe for nucleic acid intercalation;
providing the quantified single cell test imaging signal or test spectroscopy signal as an input to a machine learning classifier module utilizing a linear discriminant analysis algorithm and a training set comprising a plurality of quantified single cell imaging signals or spectroscopy signals labeled as to class of biological mechanism of action and quantified from a plurality of cells containing IR vibrational probes wherein the cells have been treated with a drug having a known class of biological mechanism of action, the IR vibrational probes comprising at least two of the following probes: a probe for protein synthesis; a probe for lipid metabolism, lipid synthesis or lipid uptake; a probe for carbohydrate synthesis; and a probe for nucleic acid intercalation, such that the machine learning classifier module determines as an output the class of biological mechanism of action of the candidate drug, or of the combination of candidate drugs; and
transmitting the class of biological mechanism of action from the machine learning classifier module to a user device and/or user display,
so as to classify a candidate drug, or a combination of candidate drugs, as having a class of biological mechanism of action at a single cell level.

A system is provided comprising:
one or more computers operatively connected to memory wherein the memory stores computer readable instructions comprising program code that, when executed, cause the one or more computers to perform the steps of:
obtaining a quantified single cell test imaging signal or test spectroscopy signal generated by a cell comprising a plurality of IR vibrational probes or Raman vibrational probes therein, wherein the plurality of different vibrational probes comprise at least two different probes which each emit a quantifiable signal in response to a different cellular parameter, wherein the cell has been treated with the candidate drug or combination;
providing the quantified single cell test imaging signal or test spectroscopy signal as an input to a machine learning classifier module utilizing a linear discriminant analysis algorithm and a training set comprising a plurality of quantified single cell imaging signals or spectroscopy signals labeled as to class of biological mechanism of action and quantified from a plurality of cells containing IR vibrational probes wherein the cells have been treated with a drug having a known class of biological mechanism of action, wherein the plurality of different vibrational probes comprise at least two different probes which each emit a quantifiable signal in response to a different cellular parameter, such that the machine learning classifier module determines as an output the class of biological mechanism of action of the candidate drug, or of the combination of candidate drugs; and
transmitting the class of biological mechanism of action from the machine learning classifier module to a user device and/or user display,
so as to classify a candidate drug, or a combination of candidate drugs, as having a class of biological mechanism of action at a single cell level.

A system is provided comprising:
one or more computers operatively connected to memory wherein the memory stores computer readable instructions comprising program code that, when executed, cause the one or more computers to perform the steps of:
providing a phenotype profile of a cell treated with said unannotated drug, or said unannotated drug combination, which phenotype profile has been obtained by a method described herein;
comparing the phenotype profile with one or more reference classes in a model obtained by applying linear discriminant analysis as a machine learning classifier to a training set of quantified single cell data, quantified from a plurality of single cells, of signals a plurality of vibrational probes comprising at least two of the following probes: a probe for protein synthesis; a probe for lipid metabolism, lipid synthesis or lipid uptake; a probe for carbohydrate synthesis; and a probe for nucleic acid intercalation, so as to provide a prediction model of a biological mechanism of action of a candidate drug, or of combination of candidate drugs, for one or more reference classes comprising protein synthesis mechanism of action; lipid metabolism mechanism of action, lipid synthesis mechanism of action, lipid uptake mechanism of action; carbohydrate synthesis mechanism of action; nucleic acid intercalation mechanism of action;
determining a Mahalanobis distance between the phenotype profile of a cell treated with said unannotated drug, or said unannotated drug combination with the one or more reference classes in the model so as to determine the reference class with a smallest Mahalanobis distance from the phenotype profile of the so-treated cell;
providing a database of drugs known to have the biological mechanism of action of said reference class with the smallest distance from the phenotype profile of the so-treated cell; and
applying an isolation forest so as to determine whether the unannotated drug or unannotated drug combination having said biological mechanism of action is novel to the class of drugs known to have the biological mechanism of action,
so as to identify a novel biological mechanism of action for an unannotated drug not previously known to have said biological mechanism of action, or for identifying a biological mechanism of action of an unannotated drug combination.

A system is provided comprising:
one or more computers operatively connected to memory wherein the memory stores computer readable instructions comprising program code that, when executed, cause the one or more computers to perform the steps of:
   providing a phenotype profile of a cell treated with an unannotated drug, or an unannotated drug combination, which phenotype profile has been obtained by a method described herein;
   comparing the phenotype profile with one or more reference classes in a model obtained by applying linear discriminant analysis as a machine learning classifier to a training set of quantified single cell signals of a plurality of vibrational probes comprising at least two different probes which each emit a quantifiable signal in response to a different cellular parameter, so as to provide a prediction model of a biological mechanism of action of a candidate drug, or of combination of candidate drugs, for one or more reference classes each of which classes comprising a mechanism of action involving at least one of the cellular parameters;
   determining a Mahalanobis distance between the phenotype profile of a cell treated with said unannotated drug, or said unannotated drug combination with the one or more reference classes in the model so as to determine the reference class with a smallest Mahalanobis distance from the phenotype profile of the so-treated cell;
   providing a database of drugs known to have the biological mechanism of action of said reference class(es) with the smallest distance from the phenotype profile of the so-treated cell; and
   applying an isolation forest so as to determine whether the unannotated drug or unannotated drug combination having said biological mechanism of action is novel to the class(es) of drugs known to have the biological mechanism of action,
   so as to identify a novel biological mechanism of action for an unannotated drug not previously known to have said biological mechanism of action, or for identifying a biological mechanism of action of an unannotated drug combination.

### BRIEF DESCRIPTION OF THE DRAWINGS

FIGS. 1A-1B: Chemical Painting workflow. (1A) Average single-cell IR spectrum of MDA-MB-231 cells with the labeling of three IR-active vibrational probes in 48 hrs. (1B) Workflow of Chemical Painting. The first step is to add drugs under its IC50 concentration together with three IR-active vibrational probes to the cell culture media, followed by cell fixation, air-drying and FTIR imaging. Next, single-cell segmentation was performed to extract single-cell IR spectrum from imaging data, forming the single-cell biochemical profiles. Downstream analysis is added to further map cell phenotypes after drug perturbations. In (3) FTIR imaging, BS represents beam splitter, MM represents moving mirror, FM represents fixed mirror.

FIGS. 2A-2C: 3D scatter plots on three metabolic activities of cells treated by various drugs. Each dot indicates the metabolic responses from an individual cell. X-axis represents saturated FA metabolism (azido-PA/CH₂), y-axis represents unsaturated FA metabolism (d₃₄OA/CH₂), and z-axis represents protein synthesis (¹³C amide I/(¹³C amide I+¹²C amide I)). Shaded areas are error ellipses with 70% confidence. (2A) Metabolic inhibitor panel from 5 different drugs inhibiting metabolism with control. ACS represents acyl CoA synthetase. FASN represents fatty acid synthase. (2B) Specific protein/metabolic pathway inhibitors panel includes 4 different drugs targeting specific protein/pathway with control. (2C) Cell responses to drugs with the same MoAs, including protein synthesis inhibitors, DNA intercalation, protein degradation inhibitors and PI3K/mTOR inhibitors. Experiments were repeated 3 times with similar results.

FIGS. 3A-3G. Comparison between multiplexed probe approach and label-free approach. (3A, 3B) Averaged single-cell spectrum of drug treatments for (3A) multiplexed vibrational probe approach and (3B) label-free approach. (3C, 3D) HCA dendrogram of cellular spectrum of multiple drug treatments for (3C) multiplexed vibrational probe approach and (3D) label-free approach. Dox represents doxorubicin, Epi represents epirubicin, Ani represents anisomycin, Cyc represents cycloheximide, Bor represents bortezomib, MG represents MG-132, Eve represents everolimus, Dac represents dactolisib, Tri represents triacsin-C, ctrl represents control group. (3E) UMAP plot of cellular spectrum of multiple drug treatments using multiplexed vibrational probe approach. UMAP plots of cellular spectrum from different batches for (3F) multiplexed vibrational probe approach and (3G) label-free approach.

FIGS. 4A-4F: Accurate prediction of drug MoAs at single-cell level. (4A, 4B) 3D dimension reduction LDA plot and confusion matrix of predictions based on LDA classifier, where PSI represents protein synthesis inhibitors, PDI represents protein degradation inhibitors, DNAI represents DNA intercalators, LFACS represents long-chain fatty acyl CoA synthetase inhibitors and FASN represents fatty acid synthase inhibitors. (4C) ROC curves of the prediction results of data from the same batch data (blue) and different batches (red). (4D) Plot of prediction accuracies versus training dataset size based on the same batch data (blue) and data from different batches (red). (4E) Feature importance ranking projected on IR spectrum with color gradient coding, where red color indicates more important features. (4F) Plot of prediction accuracies versus the number of features, based on the same batch data (blue) and data from different batches (red).

FIGS. 5A-5F: Identify test compounds with novel mechanism of action. (5A-5F) Projections of test compounds in the 3D dimension reduction LDA plot: (5A) daunorubicin; (5B) emetine, single-cell data are highly overlapped with the PSI cluster so that partial data of emetine is blocked by data from this cluster; (5C) iniparib; (5D) apicidin; (5E) taxol; (5F) vincristine. Ctrl represents the control group; DNAI represents DNA intercalations; PSI represents protein synthesis inhibitors.

FIGS. 6A-6B: Discrimination and evaluation of drug combination therapy. (6A) UMAP plot of drug combinations. Drug combination groups are highlighted in dashed circles. For Eve-Lap (1-1), the added concentration was half of the IC50 values of everolimus (14.5 μM) and half of the IC50 values of lapatinib (1.12 μM); for Eve-Lap (1-2), the added concentration was half of the IC50 values of everolimus (14.5 μM) and the IC50 values of lapatinib (2.14 μM). The Eve-Dox groups follow the same logic. (6B) Projections of drug combination data in the 3D dimension reduction LDA plot. Drug combination groups are highlighted in dashed circles. DNAI represents DNA intercalation.

FIGS. 7A-7I: Metabolic heterogeneity analysis and sub-population identification by Chemical Painting. (7A-7C) Composite plots integrating individual values and box plots of three metabolic activities from cells treated by multiple drugs: (7A) saturated FA metabolism (azido-PA/CH₂); (7B) unsaturated FA metabolism (d₃₄OA/CH₂); (7C) protein synthesis (¹³C amide I/(¹³C amide I+¹²C amide I)). (7D-7F) Cv values of corresponding three metabolic activities. The error bars include n=3 cell cultures. (7G-7I) Histograms of saturated FA metabolism with kernel density fitting in (7G) control group, (7H) cells treated by anisomycin (p-value of dip test: 0.047), (7I) cells treated by TVB-3166 (p-value of dip test: 8.40×10⁻⁵). Insets are the corresponding cumulative distribution function (CDF) of each distribution.

### DETAILED DESCRIPTION OF THE INVENTION

A method of profiling a phenotype of a cell comprising:
introducing a plurality of different infrared (IR) vibrational probes or Raman vibrational probes into the cell, wherein the plurality of different vibrational probes comprise at least two different probes which each emit a quantifiable signal corresponding to a unique cellular parameter; and
obtaining and/or quantifying, by vibrational probe spectroscopy or by vibrational probe imaging, a signal of each vibrational probe so as to determine and/or quantify presence, abundance, and/or spatial location of at least two unique cellular parameters in the cell, so as to thereby profile the phenotype of the cell.

In embodiments, the methods further comprise obtaining and/or quantifying, by spectroscopy or by imaging one or more quantifiable label-free Raman or IR vibrational signals from the cell which (i) corresponds to one or more cellular parameters and (ii) does not correspond to a vibrational probe, preferably wherein the one or more quantifiable label-free Raman or IR vibrational signals from the cell are intrinsic label-free vibration signals from a biological macromolecule within the cell.

A method of profiling a phenotype of a cell comprising:
introducing a plurality of different IR vibrational probes or Raman vibrational probes into the cell, wherein the plurality of different vibrational probes comprise at least two different probes which each emit a quantifiable signal corresponding to a unique cellular parameter; and
obtaining and/or quantifying, by vibrational probe spectroscopy or by vibrational probe imaging, a signal of each vibrational probe so as to determine and/or quantify presence, abundance, and/or spatial location of at least two unique cellular parameters in the cell, so as to thereby profile the phenotype of the cell.

In embodiments, the method further comprises obtaining and/or quantifying, by spectroscopy or by imaging one or more quantifiable label-free Raman or IR vibrational signals from the cell which (i) corresponds to one or more cellular parameters and (ii) does not correspond to a vibrational probe, preferably wherein the one or more quantifiable label-free Raman or IR vibrational signals from the cell are intrinsic label-free vibration signals from a biological macromolecule within the cell.

In embodiments the cellular parameter comprises a cell metabolism parameter.

In embodiments, the method further comprises perturbating the cell before or after profiling the phenotype of the cell.

In embodiments, the method further comprises administering to the cell, from which the phenotype is to be determined, a biological therapy or a genetic therapy, one or more drugs, candidate drugs, and/or a combination of drug treatments, prior to obtaining prior to obtaining and/or quantifying said signal(s).

In embodiments, the method comprises
introducing a plurality of different IR vibrational probes or Raman vibrational probes into the cell, wherein the plurality of vibrational probes comprises at least two of the following probes:
a probe corresponding to protein synthesis;
a probe corresponding to lipid metabolism, lipid synthesis, and/or lipid uptake;
a probe corresponding to carbohydrate synthesis; and
a probe corresponding to nucleic acid intercalation;
obtaining and/or quantifying, by vibrational probe spectroscopy or by vibrational probe imaging, a signal of each vibrational probe so as to determine and/or quantify the presence, abundance and/or spatial location of protein synthesis, lipid metabolism, lipid uptake, lipid synthesis, carbohydrate synthesis, and/or nucleic acid intercalation in the cell, so as to thereby profile the phenotype of the cell.

In embodiments, the method comprises
introducing an IR vibrational probe or Raman vibrational probe into the cell, wherein the vibrational probe comprises one of the following probes:
a probe corresponding to protein synthesis;
a probe corresponding to lipid metabolism, lipid synthesis, and/or lipid uptake;
a probe corresponding to carbohydrate synthesis; and
a probe corresponding to nucleic acid intercalation;
obtaining and/or quantifying, by vibrational probe spectroscopy or by vibrational probe imaging, a signal of each vibrational probe so as to determine and/or quantify the presence, abundance and/or spatial location of protein synthesis, lipid metabolism, lipid uptake, lipid synthesis, carbohydrate synthesis, and/or nucleic acid intercalation in the cell, so as to thereby profile the phenotype of the cell.

In embodiments, the method further comprises
i) obtaining and/or quantifying, by vibrational probe spectroscopy or by vibrational probe imaging, a signal of each vibrational probe so as to determine and/or quantify presence, abundance, and/or spatial location of at least two unique cellular parameters in the cell before and after perturbation of the cell, and
ii) comparing the
(1) determined and/or quantified at least two unique cellular parameters before the perturbation of the cell to
(2) the determined and/or quantified at least two unique cellular parameters after the perturbation of the cell, or
so as to phenotypically profile the response of the cell to the perturbation.

A method of profiling a phenotype of a cell is provided comprising:
introducing a plurality of different infrared (IR) vibrational probes or Raman vibrational probes into the cell, wherein the plurality of different vibrational probes comprise at least two different probes which each emit a quantifiable signal in response to a different cellular parameter; and
obtaining and/or quantifying, by vibrational probe spectroscopy or by vibrational probe imaging, a signal of each vibrational probe so as to determine and/or quantify presence, abundance and/or spatial location of two or more cellular parameters in the cell, so as to thereby profile the phenotype of the cell.

In embodiments, the methods further comprise obtaining and/or quantifying, by spectroscopy or by imaging one or more quantifiable Raman or IR vibrational signals from the cell not corresponding to a vibrational probe but corresponding to one or more cellular parameters.

In embodiments, the cellular parameter comprises a cell metabolism parameter. In embodiments, the quantifiable Raman or IR vibrational signals from the cell not corresponding to a vibrational probe but corresponding to one or more cellular parameters are intrinsic label-free vibration signals from biological macromolecule(s) in cells.

In embodiments, the methods further comprise administering to the cell(s), from which the phenotype is to be determined, one or more drugs, candidate drugs, or combination drug treatments.

In embodiments, the methods further comprise administering to the cell(s), from which the phenotype is to be determined, a biological therapy or genetic therapy.

In embodiments, the cellular parameter(s) is/are metabolic parameters. In embodiments, the cellular parameter(s) is/are anabolic parameters. In embodiments, the cellular parameter(s) is/are catabolic parameters. In embodiments, the cellular parameters comprise a measurable biomolecule. In embodiments, the cellular parameters comprise pH. In embodiments, the cellular parameters comprise morphology or one or more subcellular components.

In embodiments, the cellular parameters comprise one or more subcellular components.

In embodiments, the methods comprise introducing a plurality of different IR vibrational probes or Raman vibrational probes into the cell, wherein the plurality of vibrational probes comprise at least two of the following probes: a probe for protein synthesis; a probe for lipid metabolism, lipid synthesis or lipid uptake; a probe for carbohydrate synthesis; and a probe for nucleic acid intercalation; and
obtaining and/or quantifying, by vibrational probe spectroscopy or by vibrational probe imaging, a signal of each vibrational probe so as to determine and/or quantify the presence, abundance and/or spatial location of protein synthesis, lipid metabolism or lipid uptake or lipid synthesis, carbohydrate synthesis, and/or nucleic acid in the cell, so as to thereby profile the phenotype of the cell.

In embodiments, the methods further comprise:
obtaining and/or quantifying, by vibrational probe spectroscopy or by vibrational probe imaging, a signal of each vibrational probe so as to determine and/or quantify presence, abundance, and/or spatial location of at least two unique cellular parameters in the cell before and after perturbation of the cell, and
comparing the
determined and/or quantified at least two unique cellular parameters before the perturbation of the cell to
the determined and/or quantified at least two unique cellular parameters after the perturbation of the cell, or
so as to phenotypically profile the response of the cell to the perturbation.

In embodiments, the methods further comprise comparing the determined and/or quantified cellular parameter, or comparing the determined and/or quantified presence, abundance and/or spatial location of protein synthesis, lipid metabolism, lipid synthesis or lipid uptake, carbohydrate synthesis, and/or double stranded nucleic acid in the cell to a predetermined or premeasured control phenotype value for each so as to phenotypically profile the cell relative to a control phenotype.

In embodiments, the method is performed upon
i) one or more treated cells that have been subjected to a treatment, and
ii) one or more non-treated cells of the same type that have not been subjected to the treatment, wherein a predetermined or pre-measured control phenotype profile corresponds to the one or more non-treated cells,
preferably such that the method identifies any phenotypic changes that occur after the treatment based differences between the profiled phenotype of the one or more treated cells and the profiled phenotype of the one or more non-treated cells.

In embodiments, the treatment is a drug treatment or candidate drug treatment.

In embodiments, the treatment is a genetic treatment, RNAi-based therapy, gene therapy, or other genetic modification treatment.

In embodiments, the method is performed on a plurality of cells so as to identify any cell or subsets of cells of the plurality that respond differently, phenotypically, and/or have a different phenotypic profile from the remainder of the cells of the plurality, preferably to identify any cell or subsets of cells of the plurality that respond differently, phenotypically, and/or have a different phenotypic profile in response to a treatment.

In embodiments, the method comprises quantifying sample cell heterogeneity or is used to evaluate drug resistance based on the determined phenotypic profiles.

In embodiments, the nucleic acid is double-stranded. In embodiments, the nucleic acid is a DNA. In embodiments, the nucleic acid is an RNA.

In embodiments, the IR vibrational probes introduced into the cell are excited at mid-infrared wavelengths (1600-2300±200 cm⁻¹).

In embodiments, the IR vibrational probes or Raman vibrational probes introduced into the cell comprise a probe for detecting unsaturated fatty acid uptake.

In embodiments, the IR vibrational probes introduced into the cell comprise a probe for detecting unsaturated fatty acid uptake which is oleic acid-d₃₄.

In embodiments, the IR vibrational probes or Raman vibrational probes introduced into the cell comprise a probe for detecting saturated fatty acid uptake.

In embodiments, the IR vibrational probes introduced into the cell comprise a probe for detecting saturated fatty acid uptake which is azido palmitic acid.

In embodiments, the IR vibrational probes introduced into the cell comprise ¹³C labeled amino acids for detecting protein synthesis or azidohomoalanine for detecting protein synthesis.

In embodiments, the IR vibrational probes or Raman vibrational probes introduced into the cell comprise a probe for carbohydrate synthesis.

In embodiments, de novo lipid synthesis is determined using a ¹³C-glucose probe.

In embodiments, the IR vibrational probes introduced into the cell comprise a probe for intercalating double stranded DNA.

In embodiments, the methods comprise simultaneously profiling the phenotypes of a plurality of cells using the method.

In embodiments, the methods further comprise plotting the determined and/or quantified cellular parameter(s) or presence, abundance and/or spatial location of protein synthesis, lipid metabolism or synthesis or uptake, carbohydrate synthesis, and/or nucleic acid on a multi-dimensional plot so as to provide a 3D visual representation of the phenotypic profile of the cell or of the phenotypic profiles of the plurality of cells.

In embodiments, the cell is not loaded with a fluorescent dye.

In embodiments, the different IR vibrational probes used have individually resolvable absorption spectra.

In embodiments, the cell is physically isolated from other cells.

In embodiments, the cell is in contact with one or more other cells.

In embodiments, the methods further comprise chemically fixing the cell subsequent to introducing a plurality of different IR vibrational probes but prior to determining and/or quantifying the cellular parameter(s).

In embodiments, the methods further comprise chemically fixing the cell subsequent to introducing a plurality of different IR vibrational probes but prior to determining and/or quantifying protein synthesis, lipid metabolism/synthesis/uptake, carbohydrate synthesis, and/or double stranded nucleic acid in the cell.

In embodiments, the method is performed upon one or more cells that have been subjected to a treatment, and the premeasured or predetermined control phenotype corresponds to a non-treated cell of the same type, such that the method identifies any phenotypic changes that occur after the treatment.

In embodiments, the treatment is a drug treatment or candidate drug treatment.

In embodiments, the treatment is a genetic treatment, RNAi-based therapy, gene therapy or other genetic modification treatment.

In embodiments, the method is performed on a plurality of cells so as to identify any subsets of cells of the plurality that respond differently, phenotypically, to the treatment from the remainder of the cells of the plurality.

In embodiments, the method quantifies sample cell heterogeneity or is used to evaluate drug resistance.

In embodiments, the method is performed upon one or more cells that have been subjected to a candidate drug treatment, and the control phenotype corresponds to a non-treated cell of the same type, so as to identify any phenotypic changes that occur in response to the candidate drug treatment.

In embodiments, the method is simultaneously performed on more than one cell within a collection of cells.

In embodiments, the collection of cells is a subject-derived organoid.

In embodiments, the cell or cells is/are human.

In embodiments, the determining and/or quantifying is effected using vibrational probe imaging.

In embodiments, the methods comprise applying linear unmixing to signals of the vibrational probes so as to separate two or more vibrational probe signals.

In embodiments, the methods comprise performing Fourier transformed infrared (FTIR) imaging or spectroscopy on the IR vibrational probes.

In embodiments, the methods further comprise performing single-cell segmentation by masking vibrational probe imaging signals obtained.

In embodiments, the quantified vibrational probe signal is ratioed to a reference value for said vibrational probe.

A method is provided for classifying a candidate drug, or a combination of candidate drugs, as having a class of biological mechanism of action at a single cell level comprising:
obtaining a quantified single cell test imaging signal or test spectroscopy signal generated by a plurality of IR vibrational probes and quantified from a cell treated with the candidate drug or combination, the cell comprising therein a plurality of IR vibrational probes comprising at least two of the following probes: a probe for protein synthesis; a probe for lipid metabolism, lipid synthesis or lipid uptake; a probe for carbohydrate synthesis; and a probe for nucleic acid intercalation;
providing the quantified single cell test imaging signal or test spectroscopy signal as an input to a machine learning classifier module utilizing a linear discriminant analysis algorithm and a training set comprising a plurality of quantified single cell imaging signals or spectroscopy signals labeled as to class of biological mechanism of action and quantified from a plurality of cells containing IR vibrational probes wherein the cells have been treated with a drug having a known class of biological mechanism of action, the IR vibrational probes comprising at least two of the following probes: a probe for protein synthesis; a probe for lipid metabolism, lipid synthesis or lipid uptake; a probe for carbohydrate synthesis; and a probe for nucleic acid intercalation, such that the machine learning classifier module determines as an output the class of biological mechanism of action of the candidate drug, or of the combination of candidate drugs; and
transmitting the class of biological mechanism of action of the candidate drug or candidate drug combination from the machine learning classifier module to a user device and/or user display.

In some embodiments the classifying of a candidate drug is predicting the Mechanism of Action of said drug.

A method is provided for predicting the Mechanism of Action (MoA) of candidate drug, or a combination of candidate drugs, as having a class of biological mechanism of action at a single cell level comprising:
obtaining a quantified single cell test imaging signal or test spectroscopy signal generated by a plurality of IR vibrational probes and quantified from a cell treated with the candidate drug or combination, the cell comprising therein a plurality of IR vibrational probes comprising at least one of the following probes: a probe for protein synthesis; a probe for lipid metabolism, lipid synthesis or lipid uptake; a probe for carbohydrate synthesis; and a probe for nucleic acid intercalation;
providing the quantified single cell test imaging signal or test spectroscopy signal as an input to a machine learning classifier module utilizing a linear discriminant analysis algorithm and a training set comprising a plurality of quantified single cell imaging signals or spectroscopy signals labeled as to class of biological mechanism of action and quantified from a plurality of cells containing IR vibrational probes wherein the cells have been treated with a drug having a known class of biological mechanism of action, the IR vibrational probes comprising at least one of the following probes: a probe for protein synthesis; a probe for lipid metabolism, lipid synthesis or lipid uptake; a probe for carbohydrate synthesis; and a probe for nucleic acid intercalation, such that the machine learning classifier module determines as an output the class of biological mechanism of action of the candidate drug, or of the combination of candidate drugs so as to predict the Mechanism of Action; and
transmitting the mechanism of action of the candidate drug or candidate drug combination from the machine learning classifier module to a user device and/or user display.

A method is provided for method of classifying a candidate drug, or a combination of candidate drugs, as having a class of biological mechanism of action at a single cell level comprising:
obtaining a quantified single cell test imaging signal or test spectroscopy signal generated by a cell comprising a plurality of IR vibrational probes or Raman vibrational probes therein, wherein the plurality of different vibrational probes comprise at least two different probes which each emit a quantifiable signal in response to a different cellular parameter, wherein the cell has been treated with the candidate drug or combination;
providing the quantified single cell test imaging signal or test spectroscopy signal as an input to a machine learning classifier module utilizing a linear discriminant analysis algorithm and a training set comprising a plurality of quantified single cell imaging signals or spectroscopy signals labeled as to class of biological mechanism of action and quantified from a plurality of cells containing IR vibrational probes wherein the cells have been treated with a drug having a known class of biological mechanism of action, wherein the plurality of different vibrational probes comprise at least two different probes which each emit a quantifiable signal in response to a different cellular parameter, such that the machine learning classifier module determines as an output the class of biological mechanism of action of the candidate drug, or of the combination of candidate drugs; and
transmitting the class of biological mechanism of action from the machine learning classifier module to a user device and/or user display.

In embodiments, the signals comprise vibrational probe signals and spectral feature signals.

In embodiments, the training set of quantified single cell signals comprises signals from 3,000 or more single cell drug responses.

In embodiments, the methods have a classification accuracy over 90% and wherein a signal of the training set of quantified single cell data comprises 50 or more spectral features and/or the signals comprise IR vibrational probe signal features and/or non-labeled spectral features.

In embodiments, the discrete frequency infrared (DFIR) is employed to obtain quantified single cell signals.

In embodiments, the methods further comprise obtaining the quantified single cell test imaging signal or test spectroscopy signal as a phenotype profile of a cell treated with said candidate drug, or said combination of candidate drugs, by a method described herein.

A method is provided for identifying a novel biological mechanism of action for an unannotated drug, or for identifying a biological mechanism of action of an unannotated drug combination, not previously known to have said biological mechanism of action comprising: providing a phenotype profile of a cell treated with said unannotated drug, or said unannotated drug combination, which phenotype profile has been obtained by the methods described herein, comparing the phenotype profile with one or more reference classes in a model obtained by applying linear discriminant analysis as a machine learning classifier to a training set of quantified single cell signals of a plurality of vibrational probes comprising at least two of the following probes: a probe for protein synthesis; a probe for lipid metabolism, lipid synthesis or lipid uptake; a probe for carbohydrate synthesis; and a probe for nucleic acid intercalation, so as to provide a prediction model of a biological mechanism of action of a candidate drug, or of combination of candidate drugs, for one or more reference classes comprising protein synthesis mechanism of action; lipid metabolism mechanism of action, lipid synthesis mechanism of action, lipid uptake mechanism of action; carbohydrate synthesis mechanism of action; nucleic acid intercalation mechanism of action;
determining a Mahalanobis distance between the phenotype profile of a cell treated with said unannotated drug, or said unannotated drug combination, with the one or more reference classes in the model so as to determine the reference class with a smallest Mahalanobis distance from the phenotype profile of the so-treated cell;
providing a database of drugs and/or combination of drugs known to have the biological mechanism of action of said reference class with the smallest distance from the phenotype profile of the so-treated cell; and
applying an isolation forest so as to determine whether the unannotated drug or unannotated drug combination having said biological mechanism of action of said reference class.

A method is provided for identifying a novel biological mechanism of action for an unannotated drug, or for identifying a biological mechanism of action of an unannotated drug combination, not previously known to have said biological mechanism of action comprising:
providing a phenotype profile of a cell treated with said unannotated drug, or said unannotated drug combination, which phenotype profile has been obtained by the methods described herein, comparing the phenotype profile with one or more reference classes in a model obtained by applying linear discriminant analysis as a machine learning classifier to a training set of quantified single cell signals of a plurality of vibrational probes comprising at least two of the following probes: a probe for protein synthesis; a probe for lipid metabolism, lipid synthesis or lipid uptake;
a probe for carbohydrate synthesis; and a probe for nucleic acid intercalation, so as to provide a prediction model of a biological mechanism of action of a candidate drug, or of combination of candidate drugs, for one or more reference classes comprising protein synthesis mechanism of action; lipid metabolism mechanism of action, lipid synthesis mechanism of action, lipid uptake mechanism of action; carbohydrate synthesis mechanism of action; nucleic acid intercalation mechanism of action;
determining a Mahalanobis distance between the phenotype profile of a cell treated with said unannotated drug, or said unannotated drug combination, with the one or more reference classes in the model so as to determine the reference class with a smallest Mahalanobis distance from the phenotype profile of the so-treated cell;
providing a database of drugs and/or combination of drugs known to have the biological mechanism of action of said reference class with the smallest distance from the phenotype profile of the so-treated cell; and
applying an isolation forest so as to determine whether the unannotated drug or unannotated drug combination having known biological mechanism of action of known reference class.

A method is provided for identifying a novel biological mechanism of action for an unannotated drug, or for identifying a biological mechanism of action of an unannotated drug combination, not previously known to have said biological mechanism of action comprising:
providing a phenotype profile of a cell treated with said unannotated drug, or said unannotated drug combination, which phenotype profile has been obtained by the methods described herein, comparing the phenotype profile with one or more reference classes in a model obtained by applying linear discriminant analysis as a machine learning classifier to a training set of quantified single cell signals of a plurality of vibrational probes comprising at least one of the following probes: a probe for protein synthesis; a probe for lipid metabolism, lipid synthesis or lipid uptake;
a probe for carbohydrate synthesis; and a probe for nucleic acid intercalation, so as to provide a prediction model of a biological mechanism of action of a candidate drug, or of combination of candidate drugs, for one or more reference classes comprising protein synthesis mechanism of action; lipid metabolism mechanism of action, lipid synthesis mechanism of action, lipid uptake mechanism of action; carbohydrate synthesis mechanism of action; nucleic acid intercalation mechanism of action;
determining a Mahalanobis distance between the phenotype profile of a cell treated with said unannotated drug, or said unannotated drug combination, with the one or more reference classes in the model so as to determine the reference class with a smallest Mahalanobis distance from the phenotype profile of the so-treated cell;
providing a database of drugs and/or combination of drugs known to have the biological mechanism of action of said reference class with the smallest distance from the phenotype profile of the so-treated cell; and
applying an isolation forest so as to determine whether the unannotated drug or unannotated drug combination having known biological mechanism of action of known reference class.

In embodiments, the unannotated drug or unannotated drug combination having said biological mechanism of action is determined as novel to the class of drugs known to have the biological mechanism of action if it has a mean prediction score of below 0.1, wherein prediction output is ascribed 1 as inliers (known) and ascribed −1 as outliers (novel) for each single-cell data resulting from a test drug and the mean prediction score is calculated for the unannotated drug or unannotated drug combination with a dynamic range of [1, −1].

In embodiments, the methods further comprise obtaining said phenotype profile of a cell treated with said candidate drug, or said combination of candidate drugs, which phenotype profile by the methods described herein.

A method is provided for identifying differential phenotype responses to a drug among a plurality of cells from a sample comprising performing the method described herein on a sample comprising a plurality of cells, which sample has been subjected to said drug, so as to determine whether the phenotype responses to a drug among the cells of the sample are homogenous or differ, wherein non-homogeneous phenotype responses to the drug among the cells identify a differential phenotype response to the drug among the plurality of cells.

The term “cellular parameter” as used herein refers to any measurable characteristic of a cell. For example actin filament architecture, organelle size, cell shape, protein synthesis levels, carbohydrate synthesis levels, nucleic acid intercalation levels, etc.

A device is provided comprising a vibrational infrared (IR) detection device comprising a sample chamber, which vibrational IR detection device comprises a radiation source which irradiates the sample chamber with radiation of mid-infrared wavelengths (1000-3500±200 cm⁻¹) and a detector which detects and quantifies absorption spectra of a plurality of mid-infrared probes corresponding to one or more predetermined metabolic processes by a cell within a sample within said sample chamber so as to obtain spatial and quantitative data about said predetermined metabolic processes by a cell, and wherein said device presents to a user thereof the spatial and/or quantitative data from the plurality of mid-infrared probes.

In embodiments, the vibrational IR detection device comprises a FTIR spectrometer.

In embodiments, the detector comprises a photon/quantum detector or thermal detector.

In embodiments, the detector comprises an imaging sensor.

In embodiments, the spatial and/or quantitative data of different probes of the plurality is presented simultaneously and/or superimposed on an image of the cell.

In embodiments, the device comprises multiple sample chambers.

In embodiments, the device comprises multiple sample chambers as part of a multiwell plate.

In embodiments, the methods further comprise mapping the vibrational probe signal(s) to a light microscopy image or other visual image of the cell structures, so as to thereby generate a composite artificial image of the cell showing its phenotype. In embodiments, the methods further comprise generating a composite graph integrating individual values and box plots of two or more metabolic activities so as to thereby generate a composite visual graph of phenotype of the cell.

A system is provided comprising a device described herein, a biological sample in a sample chamber thereof, and a plurality of mid-infrared probes within the biological sample corresponding to one or more predetermined cellular metabolic processes.

A system is provided comprising a device described herein, comprising an irradiation source, a beam splitter, a moving mirror, and a fixed mirror.

A system is provided comprising:
one or more computers operatively connected to memory wherein the memory stores computer readable instructions comprising program code that, when executed, cause the one or more computers to perform the steps of:
obtaining a quantified single cell test imaging signal or test spectroscopy signal generated by a plurality of IR vibrational probes and quantified from a cell treated with the candidate drug or combination, the cell comprising therein a plurality of IR vibrational probes comprising at least two of the following probes: a probe for protein synthesis; a probe for lipid metabolism, lipid synthesis or lipid uptake; a probe for carbohydrate synthesis; and a probe for nucleic acid intercalation;
providing the quantified single cell test imaging signal or test spectroscopy signal as an input to a machine learning classifier module utilizing a linear discriminant analysis algorithm and a training set comprising a plurality of quantified single cell imaging signals or spectroscopy signals labeled as to class of biological mechanism of action and quantified from a plurality of cells containing IR vibrational probes wherein the cells have been treated with a drug having a known class of biological mechanism of action, the IR vibrational probes comprising at least two of the following probes: a probe for protein synthesis; a probe for lipid metabolism, lipid synthesis or lipid uptake;
a probe for carbohydrate synthesis; and a probe for nucleic acid intercalation, such that the machine learning classifier module determines as an output the class of biological mechanism of action of the candidate drug, or of the combination of candidate drugs; and
transmitting the class of biological mechanism of action from the machine learning classifier module to a user device and/or user display,
so as to classify a candidate drug, or a combination of candidate drugs, as having a class of biological mechanism of action at a single cell level.

A system is provided comprising:
one or more computers operatively connected to memory wherein the memory stores computer readable instructions comprising program code that, when executed, cause the one or more computers to perform the steps of:
obtaining a quantified single cell test imaging signal or test spectroscopy signal generated by a plurality of IR vibrational probes and quantified from a cell treated with the candidate drug or combination, the cell comprising therein a plurality of IR vibrational probes comprising at least one of the following probes: a probe for protein synthesis; a probe for lipid metabolism, lipid synthesis or lipid uptake; a probe for carbohydrate synthesis; and a probe for nucleic acid intercalation;
providing the quantified single cell test imaging signal or test spectroscopy signal as an input to a machine learning classifier module utilizing a linear discriminant analysis algorithm and a training set comprising a plurality of quantified single cell imaging signals or spectroscopy signals labeled as to class of biological mechanism of action and quantified from a plurality of cells containing IR vibrational probes wherein the cells have been treated with a drug having a known class of biological mechanism of action, the IR vibrational probes comprising at least one of the following probes: a probe for protein synthesis; a probe for lipid metabolism, lipid synthesis or lipid uptake;
a probe for carbohydrate synthesis; and a probe for nucleic acid intercalation, such that the machine learning classifier module determines as an output the class of biological mechanism of action of the candidate drug, or of the combination of candidate drugs; and
transmitting the class of biological mechanism of action from the machine learning classifier module to a user device and/or user display,
so as to classify a candidate drug, or a combination of candidate drugs, as having a class of biological mechanism of action at a single cell level.

A system is provided comprising:
one or more computers operatively connected to memory wherein the memory stores computer readable instructions comprising program code that, when executed, cause the one or more computers to perform the steps of:
obtaining a quantified single cell test imaging signal or test spectroscopy signal generated by a cell comprising a plurality of IR vibrational probes or Raman vibrational probes therein, wherein the plurality of different vibrational probes comprise at least two different probes which each emit a quantifiable signal in response to a different cellular parameter, wherein the cell has been treated with the candidate drug or combination;
providing the quantified single cell test imaging signal or test spectroscopy signal as an input to a machine learning classifier module utilizing a linear discriminant analysis algorithm and a training set comprising a plurality of quantified single cell imaging signals or spectroscopy signals labeled as to class of biological mechanism of action and quantified from a plurality of cells containing IR vibrational probes wherein the cells have been treated with a drug having a known class of biological mechanism of action, wherein the plurality of different vibrational probes comprise at least two different probes which each emit a quantifiable signal in response to a different cellular parameter, such that the machine learning classifier module determines as an output the class of biological mechanism of action of the candidate drug, or of the combination of candidate drugs; and
transmitting the class of biological mechanism of action from the machine learning classifier module to a user device and/or user display,
so as to classify a candidate drug, or a combination of candidate drugs, as having a class of biological mechanism of action at a single cell level.

A system is provided comprising:
one or more computers operatively connected to memory wherein the memory stores computer readable instructions comprising program code that, when executed, cause the one or more computers to perform the steps of:
providing a phenotype profile of a cell treated with said unannotated drug, or said unannotated drug combination, which phenotype profile has been obtained by a method described herein, comparing the phenotype profile with one or more reference classes in a model obtained by applying linear discriminant analysis as a machine learning classifier to a training set of quantified single cell data, quantified from a plurality of single cells, of signals a plurality of vibrational probes comprising at least two of the following probes: a probe for protein synthesis; a probe for lipid metabolism, lipid synthesis or lipid uptake; a probe for carbohydrate synthesis; and a probe for nucleic acid intercalation, so as to provide a prediction model of a biological mechanism of action of a candidate drug, or of combination of candidate drugs, for one or more reference classes comprising protein synthesis mechanism of action; lipid metabolism mechanism of action, lipid synthesis mechanism of action, lipid uptake mechanism of action; carbohydrate synthesis mechanism of action; nucleic acid intercalation mechanism of action;
determining a Mahalanobis distance between the phenotype profile of a cell treated with said unannotated drug, or said unannotated drug combination with the one or more reference classes in the model so as to determine the reference class with a smallest Mahalanobis distance from the phenotype profile of the so-treated cell;
providing a database of drugs known to have the biological mechanism of action of said reference class with the smallest distance from the phenotype profile of the so-treated cell; and
applying an isolation forest so as to determine whether the unannotated drug or unannotated drug combination having said biological mechanism of action is novel to the class of drugs known to have the biological mechanism of action,
so as to identify a novel biological mechanism of action for an unannotated drug not previously known to have said biological mechanism of action, or for identifying a biological mechanism of action of an unannotated drug combination.

A system is provided comprising:
one or more computers operatively connected to memory wherein the memory stores computer readable instructions comprising program code that, when executed, cause the one or more computers to perform the steps of:
providing a phenotype profile of a cell treated with an unannotated drug, or an unannotated drug combination, which phenotype profile has been obtained by a method described herein;
comparing the phenotype profile with one or more reference classes in a model obtained by applying linear discriminant analysis as a machine learning classifier to a training set of quantified single cell signals of a plurality of vibrational probes comprising at least two different probes which each emit a quantifiable signal in response to a different cellular parameter, so as to provide a prediction model of a biological mechanism of action of a candidate drug, or of combination of candidate drugs, for one or more reference classes each of which classes comprising a mechanism of action involving at least one of the cellular parameters;
determining a Mahalanobis distance between the phenotype profile of a cell treated with said unannotated drug, or said unannotated drug combination with the one or more reference classes in the model so as to determine the reference class with a smallest Mahalanobis distance from the phenotype profile of the so-treated cell;
providing a database of drugs known to have the biological mechanism of action of said reference class(es) with the smallest distance from the phenotype profile of the so-treated cell; and
applying an isolation forest so as to determine whether the unannotated drug or unannotated drug combination having said biological mechanism of action is novel to the class(es) of drugs known to have the biological mechanism of action,
so as to identify a novel biological mechanism of action for an unannotated drug not previously known to have said biological mechanism of action, or for identifying a biological mechanism of action of an unannotated drug combination.

Various inventive concepts may be embodied as a non-transitory computer readable storage medium (or multiple non-transitory computer readable storage media) (e.g., a computer memory of any suitable type including transitory or non-transitory digital storage units, circuit configurations in Field Programmable Gate Arrays or other semiconductor devices, or other tangible computer storage medium) encoded with one or more programs that, when executed on one or more computers or other processors, perform methods that implement one or more of the various embodiments described above. When implemented in software (e.g., as an app), the software code may be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

In embodiments, the methods and algorithms employed may be incapable of being performed by hand—e.g., with pen and paper.

Also, a computer may have one or more communication devices, which may be used to interconnect the computer to one or more other devices and/or systems, such as, for example, one or more networks in any suitable form, including a local area network or a wide area network, such as an enterprise network, and intelligent network (IN) or the Internet. Such networks may be based on any suitable technology and may operate according to any suitable protocol and may include wireless networks or wired networks.

Also, a computer may have one or more input devices and/or one or more output devices. These devices can be used, among other things, to present a user interface. Examples of output devices that may be used to provide a user interface include printers or display screens for visual presentation of output and speakers or other sound generating devices for audible presentation of output. Examples of input devices that may be used for a user interface include keyboards, and pointing devices, such as mice, touch pads, and digitizing tablets. As another example, a computer may receive input information through speech recognition or in other audible formats.

The non-transitory computer readable medium or media may be transportable, such that the program or programs stored thereon may be loaded onto one or more different computers or other processors to implement various one or more of the embodiments described above. In embodiments, computer readable media may be non-transitory media.

The terms “program,” “app,” and “software” are used herein in a generic sense to refer to any type of computer code or set of computer-executable instructions that may be employed to program a computer or other processor to implement various embodiments as described above. Additionally, it should be appreciated that, according to one aspect, one or more computer programs that when executed perform methods of this application need not reside on a single computer or processor but may be distributed in a modular fashion among a number of different computers or processors to implement various embodiments of this application.

Computer-executable instructions may be in many forms, such as program modules, executed by one or more computers or other devices. Generally, program modules include routines, programs, objects, components, data structures, etc. that perform particular tasks or implement particular abstract data types. The functionality of the program modules may be combined or distributed as desired in various embodiments.

Databases, if employed in the methods or devices or systems herein, may include computer readable memory (also referred to as ‘memory’). For example, data storage space 3mem IN may be and/or include computer readable memory, used to store data as described in the disclosure. Memory may be embodied by suitable hardware, including but not limited to the following: hard disk drives, serial advanced technology attachment (SATA) hard drives, SATA solid state drives (SSDs), non-volatile memory express (NVMe) SSDs, tape drives.

Also, data in databases may be stored in computer-readable media in any suitable form. For simplicity of illustration, databases may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

In embodiments, the mid-IR excitation signal results in a probe molecule vibrational signal which is converted to an electric signal via a spectroscopy device or via an imaging device.

Vibrational microspectroscopy techniques are promising for assessing single-cell drug responses by providing a biochemical fingerprint of the structure and function of cells [18]. These techniques are in principle non-destructive and contain multiplexed cellular features regarding biochemical compositions [19,20]. In general, Raman-based methods are hindered by either low speed (spontaneous Raman) [21-27], limited spectral coverage or expensive instruments (e.g., stimulated Raman) [28-31]. On the other hand, infrared (IR)-based methods usually possess higher sensitivity, larger throughput and wide spectrum coverage [32]. Although label-free IR spectroscopy has shown promise in identifying drug mechanisms in ensemble studies [18,33-40], relevant research on single-cell drug responses is largely lacking [41,42]. A pioneering study measured a few single-cell IR spectra of cells exposed to 5 different drugs in a label-free manner [41]. However, it failed to discriminate drug-treated cell spectra from control at single-cell level, likely due to insufficient specificity.

Mid-infrared (MIR) metabolic imaging was recently developed by our group [43]. By coupling MIR microscopy with IR-active vibrational probes, the metabolic specificity of this technique has been largely improved compared with the traditional label-free measure. It can achieve large-area metabolic imaging with cellular-level spatial resolution, rich metabolic information, and high throughput. The technique was applied in tissue imaging towards tissue metabolic profiling [44]. However, the utilities of MIR metabolic imaging for measuring single-cell drug responses have not been fully unleashed yet. It remains entirely unknown whether this technique has sufficient sensitivity to discriminate drug-perturbed cell phenotypes at single-cell level and there are reasons to consider that it would not work. Moreover, it is unclear as to what degree, if any, the metabolic heterogeneity of drug-treated cells can be mapped out by this technique. In addition, specific probe design, workflow and data science methods of this technique have not been achieved for single-cell drug response.

Here, we present a new method named Chemical Painting, which integrates MIR imaging, multiplexed vibrational probes and enhanced data analysis pipeline for large-scale single-cell drug response measurement. To the best of our knowledge, this is the first work that systematically studied the use of MIR metabolic imaging in single-cell drug responses and how it can be valuable for drug discovery. Several optimizations were introduced to tailor our method for measuring single-cell drug responses. Probe-wise, we introduced a new IR-active vibrational probe, deuterated oleic acid (d₃₄-OA), to monitor unsaturated fatty acid (FA) metabolism. Combining this new probe with two other probes, three-color metabolic imaging was achieved on human cancer cells. Scale-wise, we collected more than 20,000 single-cell metabolic response data corresponding to 23 different drug treatments. Analytical-wise, we introduced machine learning, novelty detection and statistical testing for single-cell data analysis.

Interesting results were discovered by Chemical Painting. First, our method is very sensitive to drug-perturbed cell phenotypes. Utilizing this property, we built a robust machine learning classifier to predict drug MoAs with high accuracy and minimal batch effects. Moreover, we designed an algorithm to identify drug compounds of novel MoA using novelty detection. This algorithm is further applied to assess drug combinations. These studies lay the foundation for applying Chemical Painting for phenotypic drug screening. In addition, through metabolic heterogeneity analysis, sub-populations of cellular phenotypes were identified during drug treatment, which can be further used to assess drug resistance. Overall, Chemical Painting has satisfied most aforementioned criteria for single-cell drug response measurements including high sensitivity, high throughput, high content, non-invasive, relatively low instrument cost and minimal batch effects. The demonstrated potential in phenotypic screening, drug resistance and combination therapy paves the way for the clinical translation of Chemical Painting toward drug discovery and development.

Vibrational microspectroscopy techniques are promising for assessing single-cell drug responses by providing a biochemical fingerprint of the structure and function of cells18. These techniques are in principle non-destructive and contain multiplexed cellular features regarding biochemical compostitions [19,20]. In general, Raman-based methods are hindered by either low speed (spontaneous Raman) [21-27]], limited spectral coverage or expensive instruments (e.g. stimulated Raman) [28-31. On the other hand, infrared (IR)-based methods usually possess higher sensitivity, larger throughput and wide spectrum coverage [32]. Although label-free IR spectroscopy has shown promise in identifying drug mechanisms in ensemble studies [18,33-40], relevant research on single-cell drug responses is largely lacking [41,42]. A pioneering study measured a few single-cell IR spectra of cells exposed to 5 different drugs in a label-free manner [41]. However, it failed to discriminate drug-treated cell spectra from control at single-cell level, likely due to insufficient specificity.

Mid-infrared (MIR) metabolic imaging was recently developed by our group [43]. By coupling MIR microscopy with IR-active vibrational probes, the metabolic specificity of this technique has been largely improved compared with the traditional label-free measure. It can achieve large-area metabolic imaging with cellular-level spatial resolution, rich metabolic information, and high throughput. However, our previous work mainly focused on the applications of this technique in tissue imaging towards tissue metabolic profiling [44]. Thus, the utilities of MIR metabolic imaging for measuring single-cell drug responses have not been fully unleashed yet. It remains unknown whether this technique has sufficient sensitivity to discriminate drug-perturbed cell phenotypes at single-cell level. Moreover, it is unclear as to what degree the metabolic heterogeneity of drug-treated cells can be mapped out by this technique. In addition, the specific probe design, workflow and data science methods of this technique have not been achieved for single-cell drug response.

Here, we present a new method named Chemical Painting, which integrates MIR imaging, multiplexed vibrational probes and enhanced data analysis pipeline for large-scale single-cell drug response measurement. To the best of our knowledge, this is the first work that systematically studied the use of MIR metabolic imaging in single-cell drug responses and how it can be valuable for drug discovery. Several optimizations were introduced to tailor our method for measuring single-cell drug responses. Probe-wise, we introduced a new IR-active vibrational probe, deuterated oleic acid (d34-OA), to monitor unsaturated fatty acid (FA) metabolism. Combining this new probe with two other probes, three-color metabolic imaging was achieved on human cancer cells. Scale-wise, we collected more than 20,000 single-cell metabolic response data corresponding to 23 different drug treatments. Analytical-wise, we introduced machine learning, novelty detection and statistical testing for single-cell data analysis.

Interesting results were discovered by Chemical Painting. First, our method is very sensitive to drug-perturbed cell phenotypes. Utilizing this property, we built a robust machine learning classifier to predict drug MoAs with high accuracy and minimal batch effects. Moreover, we designed an algorithm to identify drug compounds of novel MoA using novelty detection. This algorithm is further applied to assess drug combinations. These studies lay the foundation for applying Chemical Painting for phenotypic drug screening. In addition, through metabolic heterogeneity analysis, sub-populations of cellular phenotypes were identified during drug treatment, which can be further used to assess drug resistance. Overall, Chemical Painting has satisfied most aforementioned criteria for single-cell drug response measurements including high sensitivity, high throughput, high content, non-invasive, relatively low instrument cost and minimal batch effects. The demonstrated potential in phenotypic screening, drug resistance and combination therapy paves the way for the clinical translation of Chemical Painting toward drug discovery and development. In embodiments the method detects and uses all or many of the signals/vibrational peaks from vibrational probes as well as the intrinsic label-free vibration signals from the biological macromolecules in cells. These hundreds of features form a biochemical profile of cells that can be used for phenotyping, and makes the methods high-content since it contains hundreds of biochemical features. The methods and systems are especially useful for phenotypic screening and drug discovery.

### Results

Development of Chemical Painting for measuring large-scale single-cell drug response: Several important aspects were considered for the development of Chemical Painting, aiming at measuring single-cell biochemical responses during drug treatments. First, for cell line selection, human cancer cell lines represent the cancer of origin and are widely used for anticancer drug screening [45,46]. Here, metastatic breast cancer cell line MDA-MB-231 was chosen as the model cell line to mimic the drug screening process.

In addition, probe design is crucial for Chemical Painting to further improve its metabolic specificity, considering that label-free approach failed to identify cell responses of drugs at single-cell level [41]. Several layers of requirements are expected for probe design. These probes should reflect different metabolic activities and be able to work together rather than against each other. Moreover, their signals should be captured simultaneously and distinguishable by MIR imaging. In our previous work, we demonstrated the separate use of two IR-active vibrational probes, ¹³C amino acids (¹³C-AA) and azido-palmitic acid (azido-PA), to reflect protein synthesis and saturated FA metabolism, respectively. Here we introduced a new IR-active vibrational probe, deuterated oleic acid (d₃₄-OA), capable of reporting unsaturated FA metabolism. To the best of the inventors' knowledge, this is the first time that d₃₄-OA has been utilized as a vibrational probe in MIR imaging. Combining this new probe with the reported probes, 3-color metabolic imaging can be achieved to report three distinctive metabolic activities. It is important that the captured metabolic activities, including protein synthesis, saturated FA metabolism and unsaturated FA metabolism, are essential metabolic processes necessary to support the survival and functions of cells. As long as the disease mechanism has a projection on major metabolic pathways, the corresponding drug candidate or drug MoA is likely to be identified by our method.

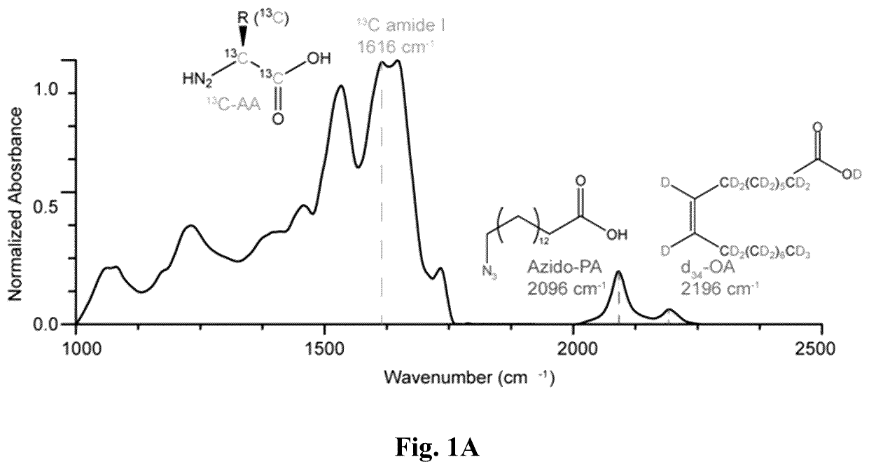

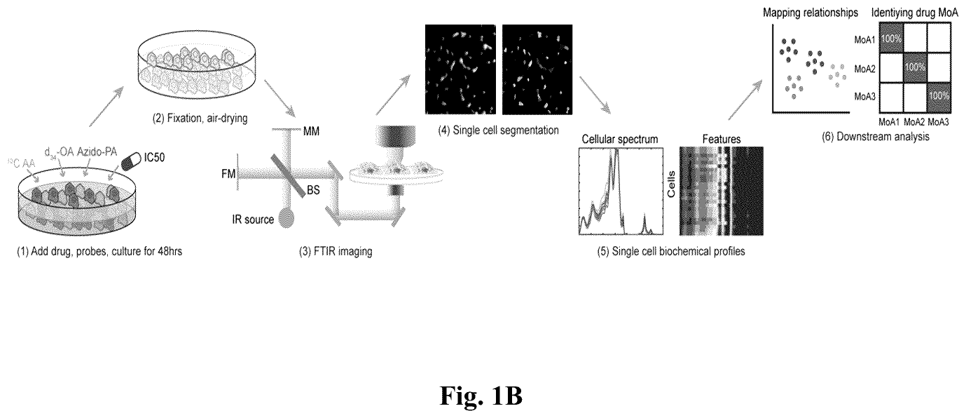

The average single-cell IR spectrum of cells co-cultured with these three IR-active vibrational probes for 48 hrs is shown in FIG. 1a. The culturing time was selected based on the trade-off between metabolic labeling signal and experimental time. It can be observed that the signals of these three probes are captured simultaneously and spectrally separable. The red-shifted amide I band at 1616 cm-1 corresponds to the newly synthesized proteins from ¹³C-AA labeling. The 2096 cm-1 peak mainly stems from the signal of azide bonds in azido-PA. The new probe, d₃₄-OA has two separate peaks at 2092 cm-1 and 2196 cm-1 in the cell-silent region of IR spectrum corresponding to asymmetric and symmetric CD2 vibrations, respectively. Noteworthy, although the azido-PA and d₃₄-OA have overlapped absorbance at 2096 cm-1, the unique peak of d₃₄-OA at 2196 cm-1 can be used to reflect the concentrations of d₃₄-OA uptake in cells. To obtain pure signals from azido-PA, linear unmixing was used to readily separate the signal of azido-PA from d₃₄-OA (Methods).

Another aspect to consider is how to ensure the cells are in a similar state under different categories of drug treatments. To achieve this, we measured the half maximal inhibitory concentration (IC50) of different drugs separately by cell viability assays in 48 hrs, in agreement with the probe culturing time. This method is commonly used in pharmaceutical studies to compare the effect of various products [39,47]. Besides the experimental operations, data analysis is vital to the obtained large-scale data to map cell phenotypes after drug perturbation. We thus built a complete analytical pipeline for Chemical Painting, including data preprocessing, single-cell segmentation, single-cell biochemical profile extraction and downstream analysis.

The complete workflow of Chemical Painting is presented in FIG. 1b. The first step is to add the three vibrational probes and the tested drug at its IC50 concentration to the cell culture media and co-cultured for 48 hrs. After that, cells are fixed and air-dried. Then Fourier transformed infrared (FTIR) imaging, a modality of MIR imaging, was performed to collect the full spectral data of cells. The collection speed is around 2 cells/second for human cancer cells with sufficient co-scans (˜64 scans). In the following data analysis, quality test, baseline correction and data normalization were included for data preprocessing (Methods). Next, single-cell segmentation was performed with open-source software CellProfiler [48] to generate masks. These masks can be further used to extract single-cell IR spectrum from the MIR imaging data, forming the single-cell biochemical profile. We then introduced a series of analytical methods for the downstream analysis, including machine learning, novelty detection and statistical methods to map cell phenotypes and identify drug MoAs. Overall, the workflow of Chemical Painting is straightforward in experimental operations and effected in single-cell data analysis, which laid the foundation for this method to reveal valuable insights toward phenotypic profiling and potential drug discovery.

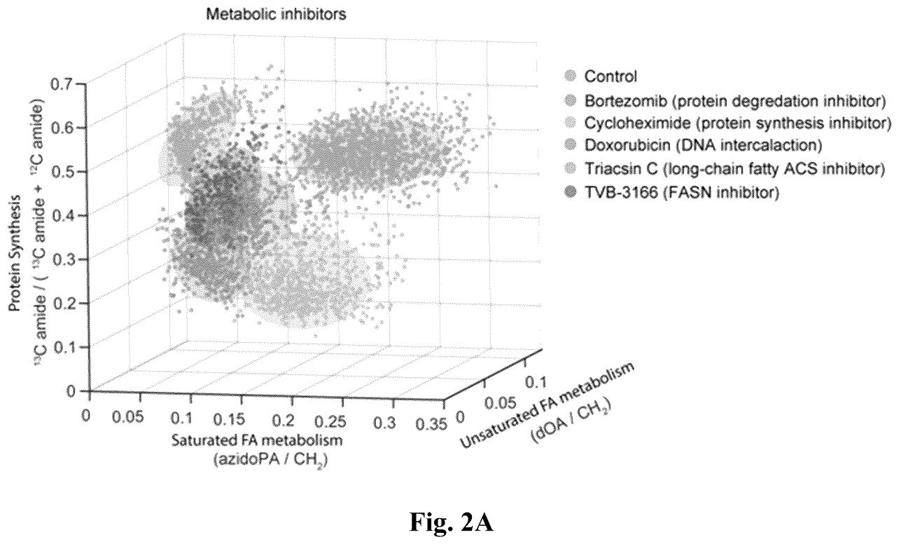

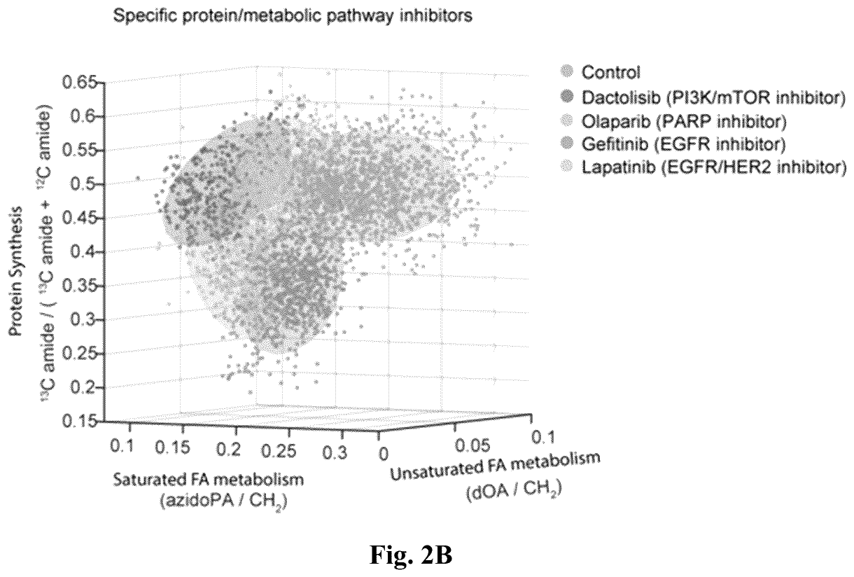

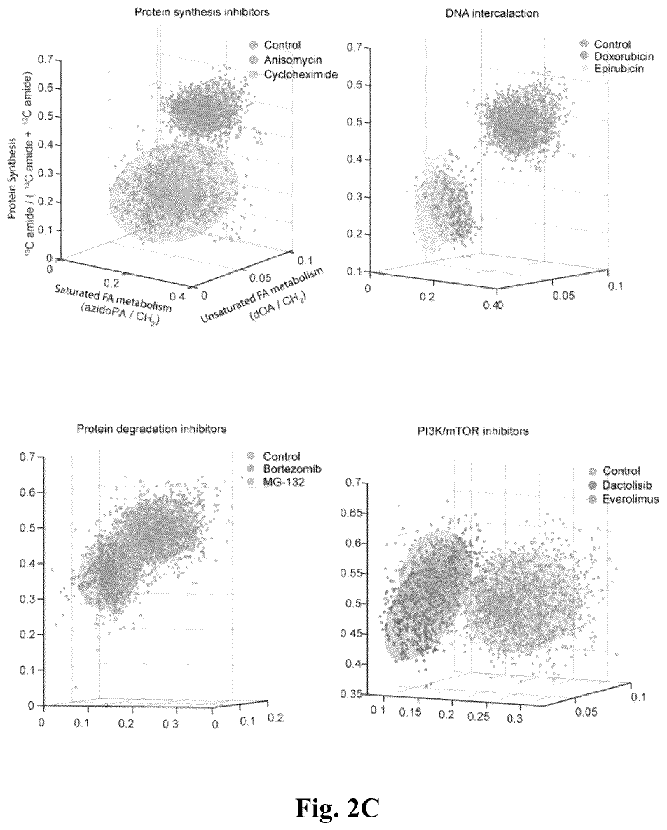

Systematic sensitivity and generalizability evaluation of Chemical Painting toward a broad range of drugs: It remains unknown whether our method has sufficient sensitivity to differentially discriminate cell responses to different drugs. To evaluate its sensitivity we tested 13 drugs belonging to 9 different drug MoAs. These drugs can be separated into two main panels: one consists of drugs that inhibit general metabolism; another one contains drugs targeting specific proteins or interfering with particular metabolic pathways. The single-cell metabolic responses toward different drug treatment were visualized in the 3D scatter plots where x-axis represents saturated FA metabolism (azido-PA/CH₂), y-axis represents unsaturated FA metabolism (d₃₄OA/CH₂), and z-axis represents protein synthesis (¹³C amide I/(¹³C amide I+¹²C amide I)). The use of ratios is meant for more accurate quantification of metabolic activities. For each drug treatment, around 500-1000 single-cell data were analyzed and presented in FIG. 2. Each dot represents the metabolic responses of an individual cell in the plots.

In the metabolic panel (FIG. 2a), five drugs that inhibit known metabolism were selected to validate our method's ability to report protein synthesis and lipid metabolism. They were also used to assess the sensitivity of our method toward drugs targeting general metabolism. Remarkably, cells treated with drugs of different MoAs form identifiable clusters, demonstrating our method's sensitivity toward diverse metabolic inhibitors. For instance, cells exposed to triacsin-C (inhibits lipid metabolism) showed significant decreases in both saturated and unsaturated FA metabolisms, whereas the cell protein synthesis remained at a similar level compared with the control group. This result indicates the ability of our method to specifically probe lipid metabolism. Additionally, cells treated by cycloheximide, which inhibits protein synthesis, displayed much lower protein synthesis ratios, showing the capability of our method to reflect protein synthesis. Interestingly, cells treated with doxorubicin showed both FA metabolisms and protein synthesis as largely inhibited. This is consistent with the mechanism of doxorubicin interfering with DNA replication and inhibiting multiple macromolecule biosynthesis [49,50]. For TVB-3166 and bortezomib, all three metabolic activities are reduced to different levels. For TVB-3166 (an FA synthase inhibitor), FA metabolism was significantly reduced while protein synthesis was slightly decreased. Whereas for bortezomib (protein degradation inhibitor), protein synthesis was more evidently decreased. Together, these varied responses under different drug treatments demonstrated the sensitivity of our method in identifying potential drug candidates for inhibiting general metabolism.

We further tested drugs that target specific proteins or that interfere with particular metabolic pathways, including dactolisib (a dual PI3K/mTOR inhibitor), olaparib (a PARP inhibitor), gefitinib (a EGFR inhibitor) and lapatinib (a dual EGFR/HER2 inhibitor). These drugs were selected due to their reported therapeutic effects on breast cancer cell lines [51]. Remarkably, cells treated with these different drugs also exhibited distinctive metabolic responses (FIG. 2b), indicating the sensitivity of our approach toward drugs targeting specific proteins or metabolic pathways. For olaparib, all three metabolic activities were significantly reduced compared to the control group. This is consistent with its PARP inhibition mechanism, which impedes the repair of single-stranded DNA breaks and further affects various macromolecule metabolism [52]. Considering this drug has been under phase-III FDA clinical trial and recently approved for the adjuvant treatment of breast cancer [53], the demonstrated decrease of metabolic activities echoes its promising therapeutic effect. The other three drug groups tested also demonstrated different metabolic activities. It is interesting to observe that our approach can differentiate gefitinib (an EGFR inhibitor) and lapatinib (an EGFR/HER2 inhibitor), which further supports the sensitivity of our method to discriminate different drug MoAs.

To evaluate the generalizability of our method, we selected drugs belonging to the same MoA and analyzed their metabolic activities (FIG. 2c). Interestingly, drugs with the same MoA display very similar metabolic responses. This finding is valid for both drugs inhibiting general metabolism (protein synthesis inhibitors, DNA intercalators, protein degradation inhibitors) and drugs inhibiting specific proteins or metabolic pathways (PI3K/mTOR inhibitors). Moreover, we tested the influence of batch effects on the signals from metabolic vibrational probes. Cells from different batches treated by the same drug are highly overlapping in the 3D scatter plots. Together, these results demonstrate the sensitivity and generalizability of our method in discriminating drug MoA with minimal batch effects, which lays the foundation of Chemical Painting to identify drug MoAs.

Comparison between multiplexed vibrational probe approach and a label-free approach: Despite the high sensitivity and generalizability demonstrated by Chemical Painting, it remains unknown whether the use of multiplexed vibrational probes is indeed advantageous compared with a label-free approach in characterizing drug responses. We thus conducted systematic comparison of these two approaches.

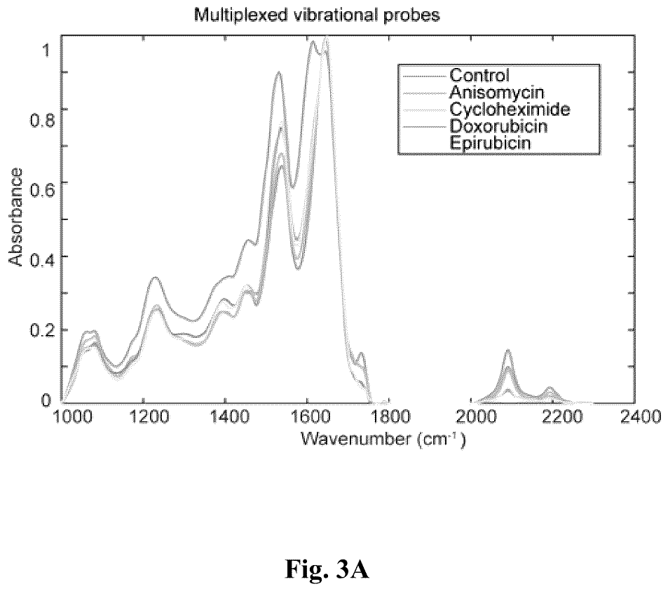

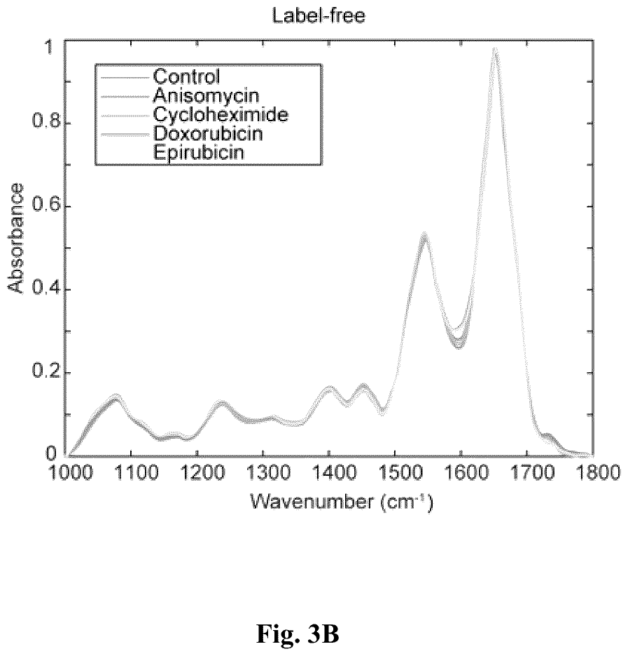

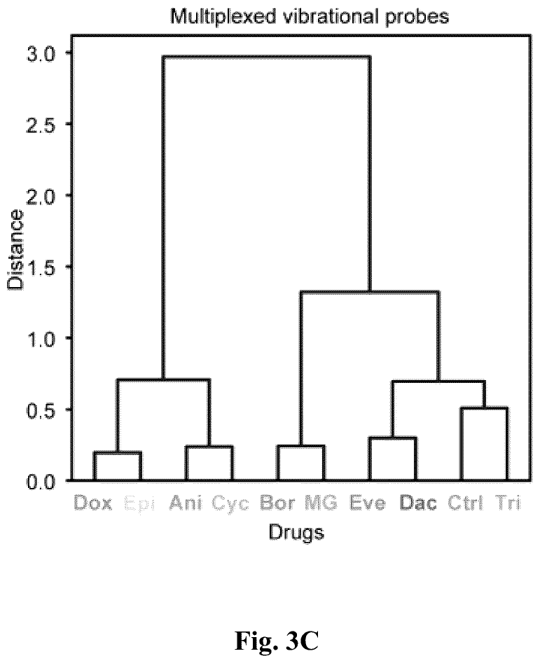

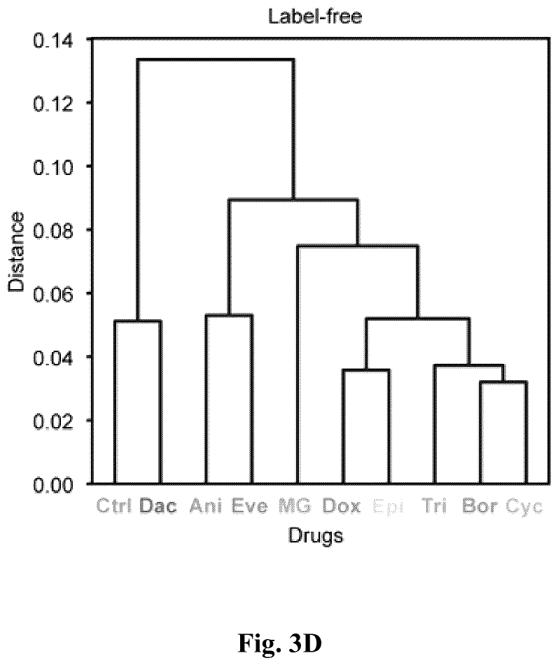

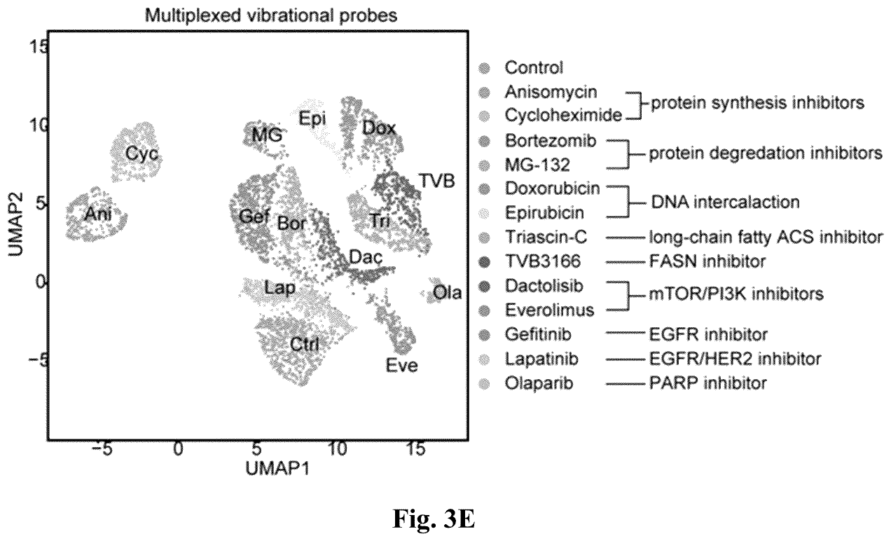

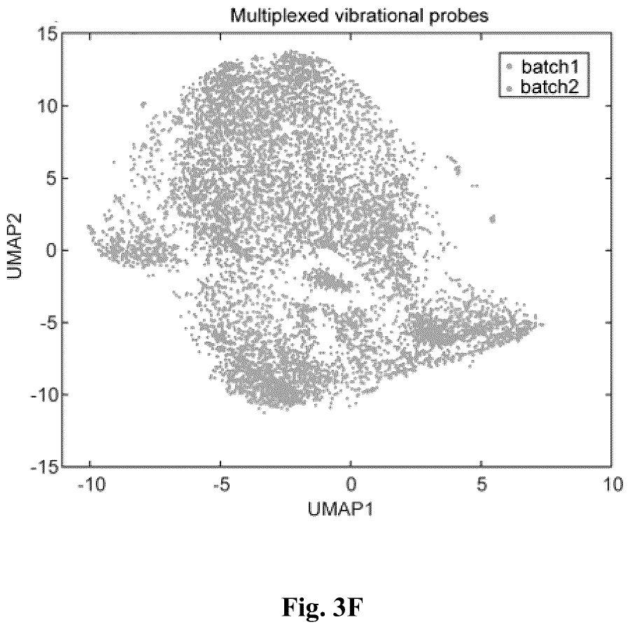

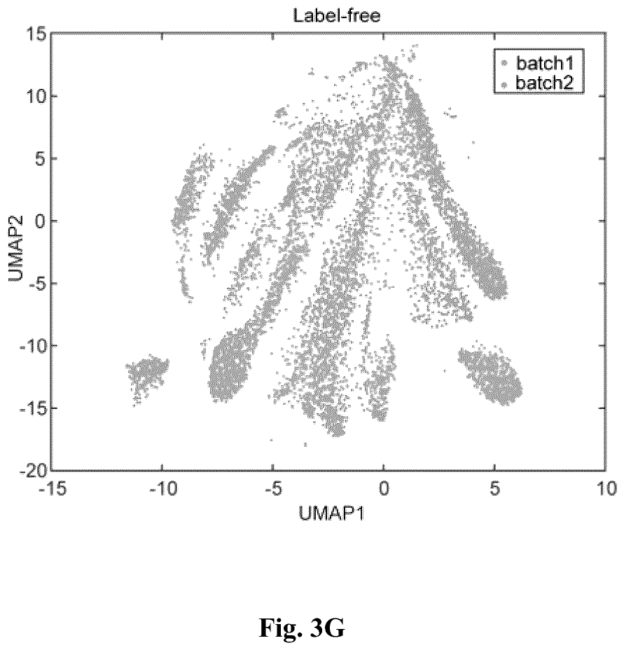

We first extracted the average single-cell spectrum of cells treated by drugs belonging to two different MoA: (1) protein synthesis inhibitors (anisomycin, cycloheximide) and (2) DNA intercalation (doxorubicin, epirubicin) (FIG. 3a, 3b). For the probe approach, it was observed that the cellular spectra of drugs with different MoAs are clearly distinct from each other, and separable from the control group. Moreover, the cellular spectra of drugs within the same MoAs (e.g., doxorubicin and epirubicin) are very similar. However, for the label-free approach, the cellular spectra under different conditions were highly overlapping. These results directly showed the ability to distinguish different drug MoAs with the use of multiplexed vibrational probes.

Next, we performed hierarchical clustering analysis (HCA) on cellular spectra of multiple drug treatments using these two approaches (FIG. 3c, 3d). For the probe approach, the dendrogram clearly grouped drugs within the same MoAs together, as coded by similar colors (e.g., anisomycin and cycloheximide). This result is consistent with the previous finding that drug within the same MoAs share similar metabolic responses (FIG. 2c). Moreover, it is further confirmed in the related Uniform Manifold Approximation and Projection (UMAP) [54] plot (FIG. 3e), where drugs within the same MoAs are distributed closer to each other and drugs with different MoAs are well-separated and from distinct clusters. The label-free approach dendrogram does not show this trend, with anisomycin and cycloheximide are located far away from each other. The ability to group drugs with the same MoAs together is a very important aspect in phenotypic drug discovery, known as the guilt-by-association approach to determine the drug MoA3. Drugs with similar MoAs should generate similar phenotypic signatures for identification. The superior performance of the probe approach in this aspect further shows the advantage of using multiplexed vibrational probes in Chemical Painting for phenotypic drug discovery.

We further evaluated batch effects of these two approaches, which is important in measuring single-cell drug responses to provide reliable readouts. The corresponding UMAP plots of drug-perturbed cellular spectra from different batches are shown in FIG. 3f-3g. For the probe approach, data from different batches are well mixed together, indicating minimal batch effects. This result is also consistent with the overlapped metabolic responses for data from different batches. In contrast, for the label-free approach, data from two batches clearly form two separate branches, suggesting severe batch effects in this approach. Overall, our studies demonstrated three advantages of using multiplexed vibrational probes compared with the label-free approach: improved sensitivity in discriminating between different drugs; improved generalizability to identify drug MoAs; and reduced batch effects.

Accurate prediction of drug MoAs at single-cell level using machine learning: Phenotypic screening has been tremendously powerful for identifying novel small molecules as probes and potential therapeutics [45]. Compared with traditional target-based approaches, the advantage of phenotypic screening is to identify drug leads and clinical candidates that are more likely to possess therapeutically relevant drug MoAs, without concerning their target-binding affinity [55]. The results here show clear separation of drug MoAs in HCA clustering and UMAP plots, with drugs of same MoAs grouped together using multiplexed vibrational probes.

To perform classification and predictions of drug MoAs, we used the cellular spectra of thirteen different drug treatments plus a control group as the dataset (from the probe approach). This dataset contains around 6,000 single-cell data and 288 features (vibrational peaks in 1000-1800 cm-1, 2000-2300 cm-1). Cells exposed to drugs with the same MoA belong to the same class (10 different classes: 9 MoAs plus the control group). The dataset was split into 70% for training and 30% for testing. We first evaluated the prediction performances using multiple popular machine learning methods (Table 1). It can be observed that many classifiers, such as linear discriminant analysis (LDA), multi-layer perceptron (MLP), random forest (RF) and XGBoost, have extremely high accuracies (over 99%) to predict drug MoAs at single-cell level. Moving forward, we further tested the influence of batch effects on these classifiers, which are important in practical drug screening. The ideal situation is that the chosen method provides stable readouts over different batches. Our testing strategy was to use classifiers trained on the same batch data to directly predict data collected from different batches, which ensures models are generalizable to batch effects. Interestingly, only LDA has high prediction accuracies on the dataset from different batches (Table 1). The decreased accuracy in other classifiers might be caused by overfitting due to model complexity.

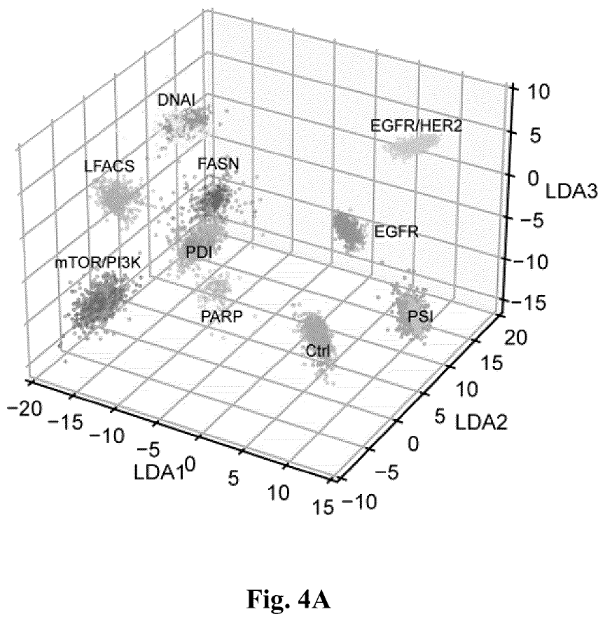

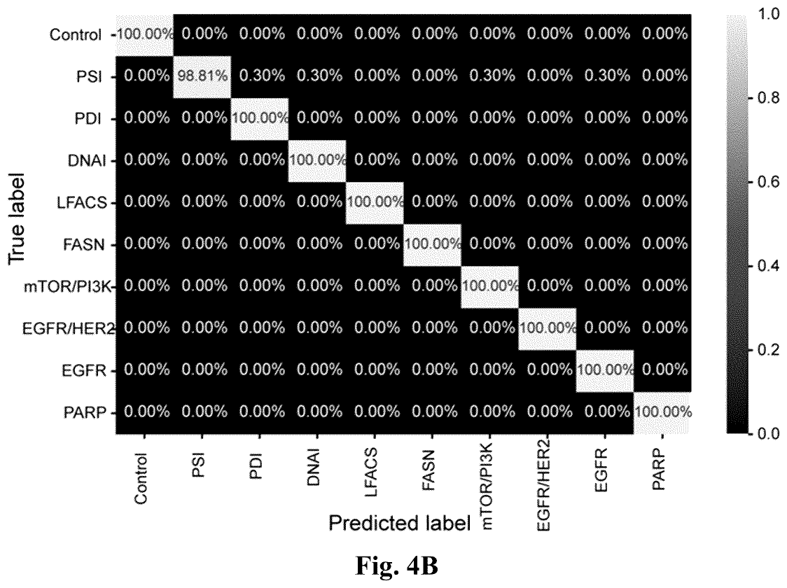

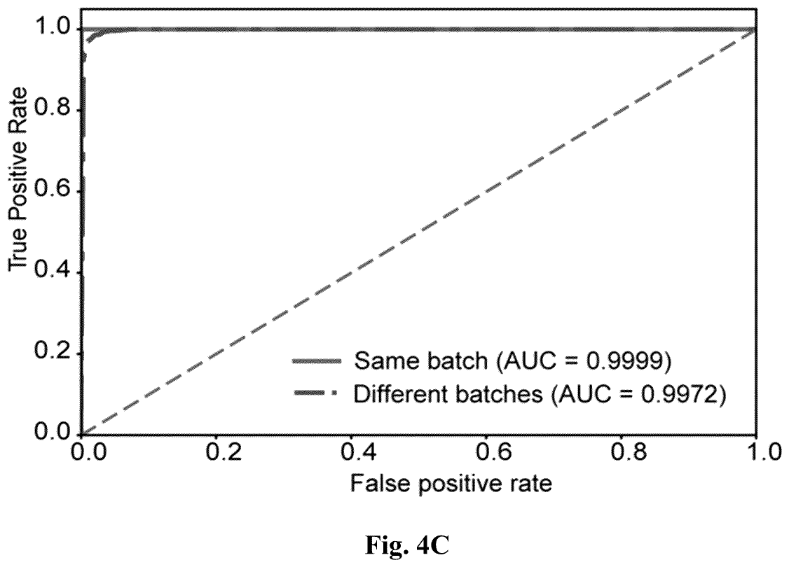

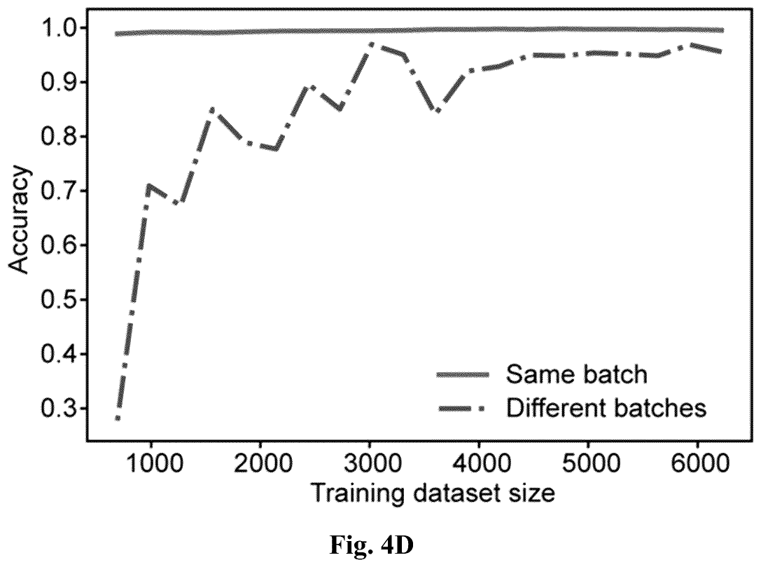

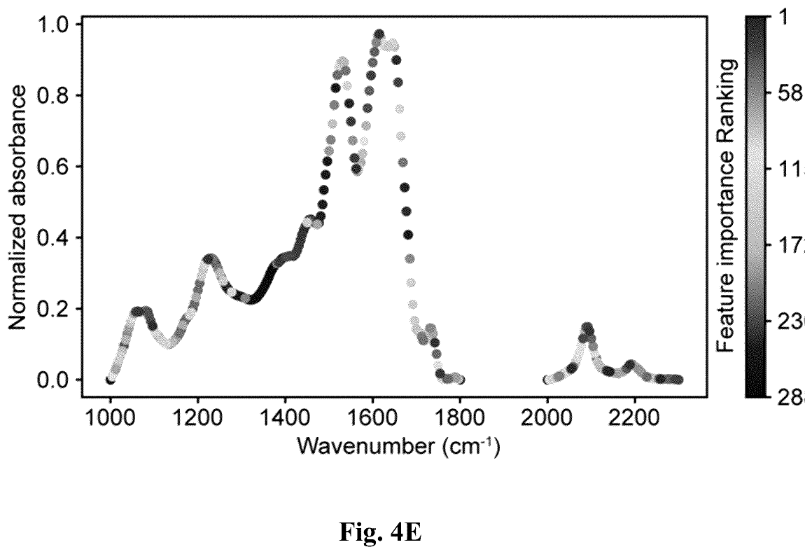

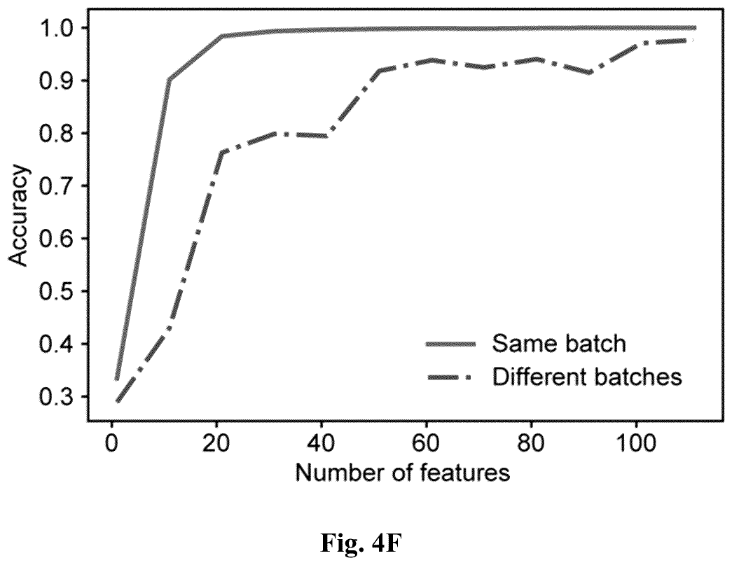

We thus selected LDA as the machine learning classifier to predict drug MoAs. The 3D LDA dimension reduction plot is shown in FIG. 4a. It can be observed that the single-cell data under different drug MoAs formed distinct clusters. This result agrees well with the high prediction accuracy achieved by LDA. The corresponding confusion matrix is shown in FIG. 4b (based on the same batch data). It can be seen that the prediction accuracy of individual drug MoA is also very high (nearly 100%). Additionally, the ROC curve is presented both for the predictions on data from the same batch and different batches (FIG. 4c), to further showcase the accurate prediction results from our method. It turns out that the dataset size plays an important role. Although high accuracy prediction can be achieved within 1000 single-cell drug responses for data from the same batch, a dataset size above 3000 is needed to achieve accuracy over 90% on data from different batches (FIG. 4d). This finding suggests the necessity of collecting large-scale single-cell data for accurate prediction.

Further studies on features reveal important insights. To achieve high prediction accuracy (over 90%) overcoming batch effects, the required number of features is around 50 (FIG. 4f). This result is valuable for further applying our method to discrete frequency infrared (DFIR) imaging [56], whose throughput can be further improved to 10-100 times by using quantum cascade lasers with selected frequency imaging. To understand the relative importance of these spectral features in drug MoA prediction, the ranking of permutation feature importance was plotted in the average IR spectrum (FIG. 4g). Color gradient coding was used for better visualization, where red colors represent more important features. It can be observed that the features coming from vibrational probes (1600-1616 cm-1, 2000-2300 cm-1) are highlighted by deep red colors. We also utilized the intrinsic feature importance in RF classifier, which shows a similar pattern. These results suggest that features arising from metabolic labeling play more important roles in predicting drug MoAs. In control experiments, we further tested whether single-cell spectrum measured in the label-free approach is sufficient to predict drug MoAs. Although the average accuracy is high for label-free data from the same batch using LDA classifier, it drops dramatically to only 21.12% after including data from different batches. This further confirmed the advantage of the probe approach in drug MoA prediction.

Overall, we systematically studied the possibility of Chemical Painting to predict drug MoAs using biochemical profiling. A reliable classifier (LDA) with stable and superb prediction performances across different batches was selected. The trained model can be used as an initial reference for further screening applications. This study demonstrates the tremendous potential of our method in phenotypic drug screening.

TABLE 1
Average accuracy of machine learning classifiers for drug MoA prediction at single-cell level:

| | DT | LDA | KNN | MLP | NB | QDA | RF | SVM | XGBoost |
|---|---|---|---|---|---|---|---|---|---|
| Same Batch | 94.02% | 99.81% | 90.93% | 99.71% | 86.73% | 91.61% | 99.08% | 97.05% | 99.42% |
| Different Batches | 31.09% | 94.79% | 47.00% | 36.30% | 61.85% | 61.91% | 37.36% | 43.23% | 35.46% |

Note:
DT represents decision tree; LDA represents linear discriminant analysis; KNN represents k-nearest neighbors; MLP represents multi-layer perceptron; NB represents naïve bayes; QDA represents quadratic discriminant analysis; RF represents random forest; SVM represents support vector machine; XGBoost represents extreme gradient boosting.

Discover drugs with novel MoAs using novelty detection: The ability to identify the MoA of lead compounds as either known or novel is crucial in phenotypic drug screening [57]. By evaluating the similarity of unannotated compounds to annotated drugs, it can either elucidate the MoA of test compounds as a known MoA or discover compounds with completely novel MoAs. This is known as a strong advantage of phenotypic profiling methods in drug discovery [58].

We specifically designed an algorithm for Chemical Painting to identify drugs with novel MoAs, using the dataset and model in drug MoA prediction as an annotated reference. In this algorithm, the first step is to calculate the Mahalanobis distance between single-cell data from test compound and each reference class in the trained LDA model. The class with the minimum distance represents the known drug closest to the test compound. Next, isolation forest [59], is introduced to determine whether the test compound is novel to the selected known drug class. The prediction output in this step is 1 as inliers (known) and −1 as outliers (novel) for each single-cell data from the test compound. Integrating the prediction results from all the single-cell data, a mean prediction score can be calculated for the test compound with a dynamic range of [1, −1], where score of 1 represents all the single-cell data of the test compound are predicted as inliers (known) and −1 represents the opposite. We thus selected mean score of 0.1 as the threshold value, which suggests 60% percent of single-cell data are predicted as inliers (known). Test compound with a mean prediction score below the threshold value is detected as novel.

The analytical results of 6 tested compounds are shown in Table 2. For compounds belonging to known mechanism in the reference, such as daunorubicin (DNA intercalation) and emetine (protein synthesis inhibitor), their mean prediction scores are high with correct prediction of drug MoAs. Whereas, for compounds with novel mechanisms, their mean prediction scores are quite negative, suggesting the high probability of these compounds being novel. These results showed the feasibility of our algorithm to identify drugs having a novel MoA.

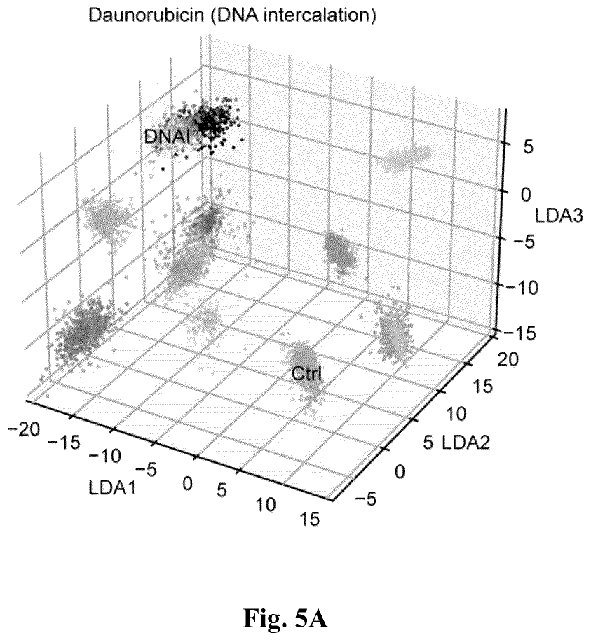

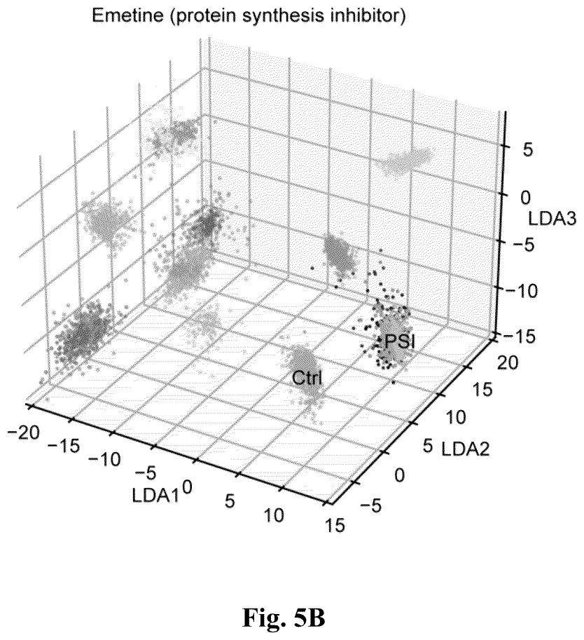

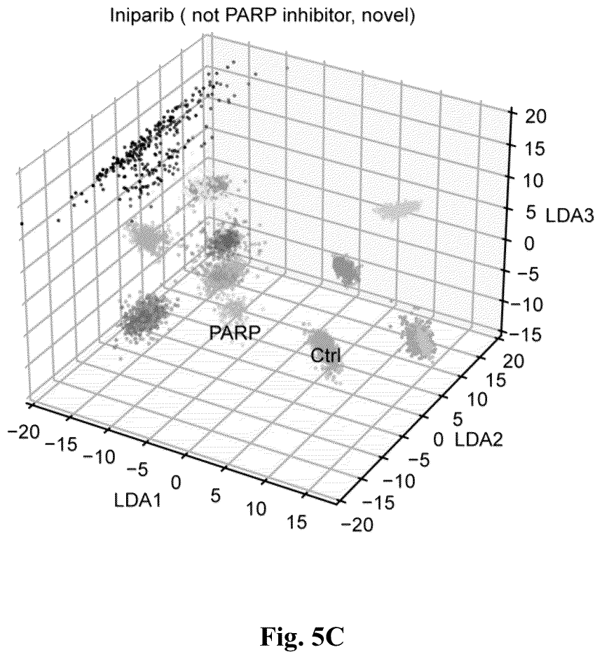

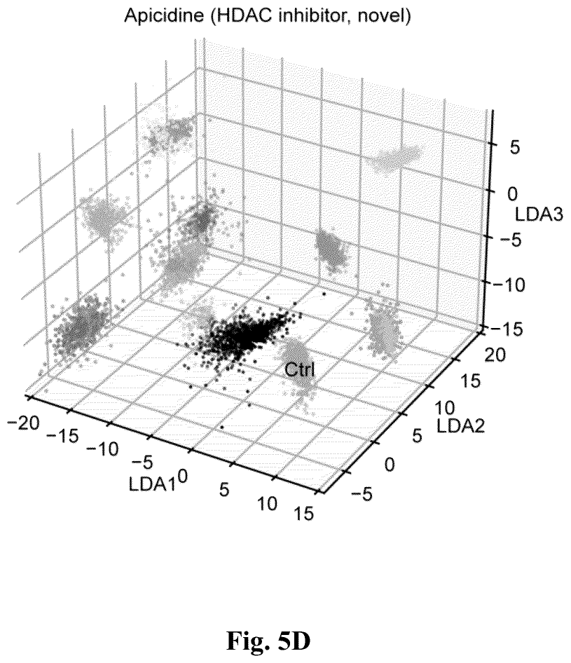

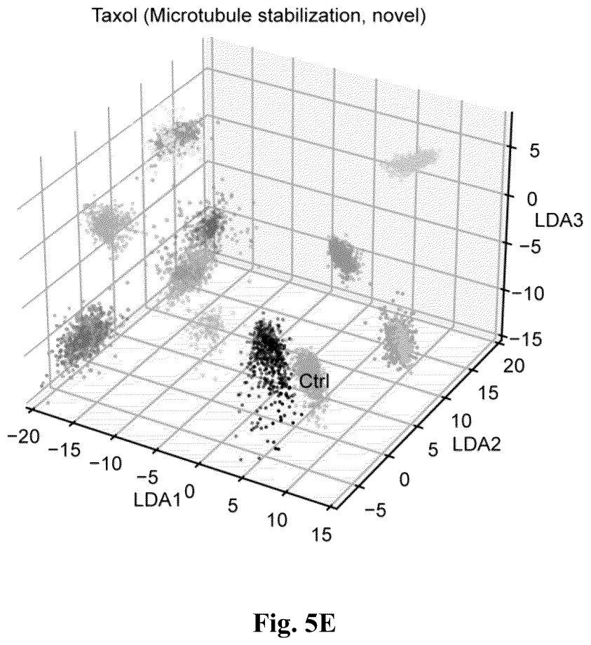

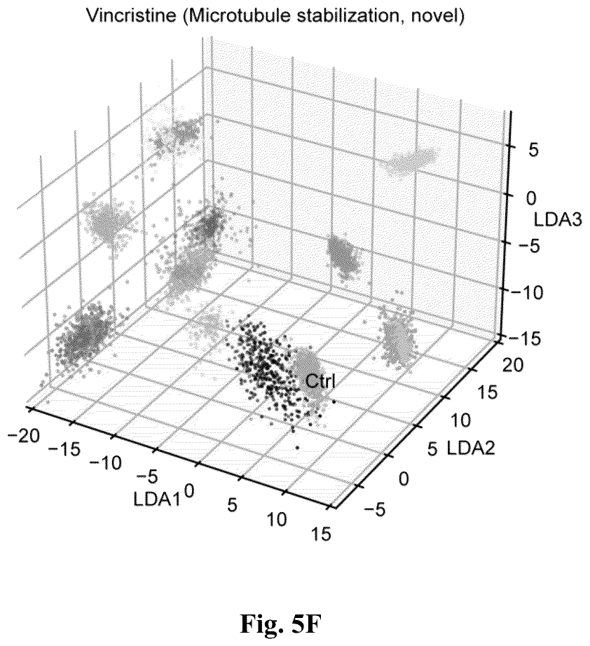

To validate the prediction results, we plotted the projections of test compound data in the LDA-reduced dimensions calculated from the reference (FIG. 5). Data from test compounds are highlighted in black colors. It can be observed that for compounds with known drug MoAs, their projected data is closely distributed to the corresponding drug MoAs (FIG. 5a, 5b). While for compounds of novel MoAs, they generally form a new cluster (FIG. 5c-f). These results agree well with the novelty prediction result from our algorithm. An interesting finding is that for taxol and vincristine, which share the same MoA, their distributions are quite similar in the projection plots (FIG. 5e, 5f). This further indicates the ability of our method to identify drug with similar MoAs. Another interesting finding is about iniparib, which was initially suggested as PARP inhibitor and went-through FDA clinical trials treating breast cancer [60], but eventually failed at Phase III. Later experiments proved that iniparib is not a PAPP inhibitor at all and its MoA is still unknown today [61]. Our result agrees with the later experiments, where iniparib has a quite negative mean prediction score and distributes far away from PARP inhibitor cluster in the LDA plot (FIG. 5c). This result further demonstrated the ability of our method to screen real novel compounds. Overall, the designed algorithm, which integrates LDA and novelty detection, is found reliable in determining compounds with known MoA or novel MoA in Chemical Painting.

TABLE 2
Prediction of drug MoAs as known or novel using novelty detection:

| Drugs | Mechanism of Action | Mean score | Outlier % | Prediction |
|---|---|---|---|---|
| Daunorubicin | DNA intercalation | 0.8497 | 7.515% | DNA intercalation |
| Emetine | Protein synthesis inhibition | 0.6208 | 18.96% | Protein |
| | | | | synthesis inhibition |
| Iniparib | Unknown | −0.9923 | 99.62% | Novel |
| Apicidin | HDAC inhibitor | −0.5289 | 76.45% | Novel |
| Taxol | Microtubule stabilization | −0.5052 | 75.26% | Novel |
| Vincristine | Microtubule stabilization | −0.9466 | 97.33% | Novel |

Discrimination of drug combinations: Combination therapy, a treatment modality that combines two or more therapeutic agents, has become a cornerstone of cancer therapy [62]. It provides a complementary strategy to new drug discovery, and can enhances the efficacy of chemotherapeutics. It can provide synergistic or additive anti-cancer effects and potentially reduce drug resistance [62]. To explore whether our method can further assess drug combinations, we used two different drug combinations under FDA clinical trials for breast cancer as technical demonstrations [63]: (i) everolimus and lapatinib (Eve-Lap) and (ii) everolimus and doxorubicin (Eve-Dox). The synergy of drug combinations was determined using cell viability assay with an open-source package SynergyFinder [64]. For each drug combination, we selected two drug concentration groups with high synergy scores, including Eve-Lap (1-1), Eve-Lap (1-2), Eve-Dox (2-1) and Eve-Dox (1-2). The groups were named based on their concentration with respect to half of the IC50 values from single drug treatments.

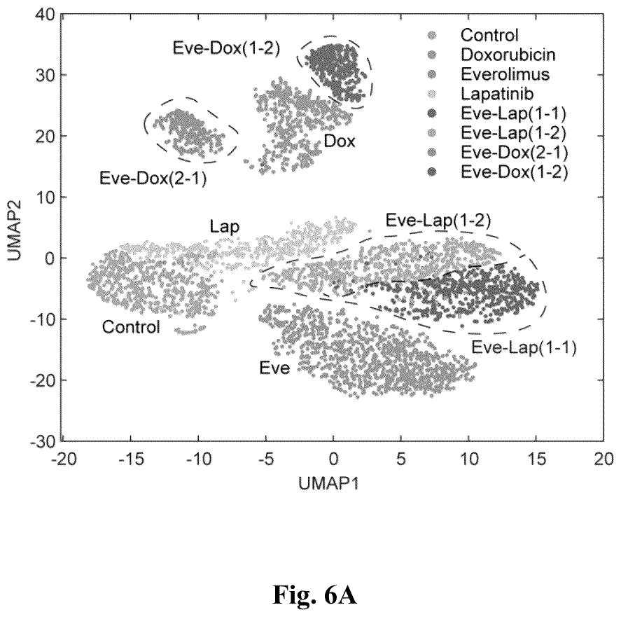

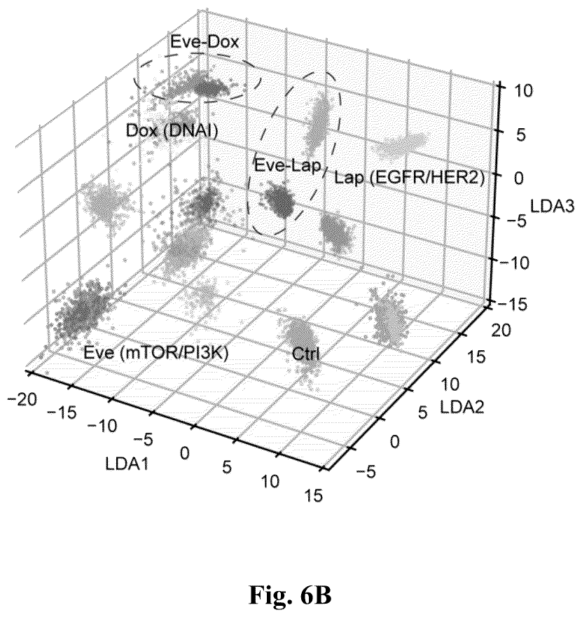

To further test whether our method can discriminate drug combinations from single drug treatment, we first used UMAP to better visualize their data distributions (FIG. 6b). Remarkably, we found that the drug combination groups form separable clusters in the plot. Moreover, they are distributed exactly between single drug treatments, which agrees well with their MoAs. For instance, both Eve-Lap (1-1) and the Eve-Lap (1-2) groups lie between the everolimus and lapatinib (single drug treatment), with Eve-Lap (1-1) closer to the everolimus cluster and Eve-Lap (1-2) closer to the lapatinib cluster, consistent with their concentration ratios. The distributions of Eve-Dox groups are also between single drug treatments but closer to the doxorubicin group. The UMAP plot qualitatively demonstrated the ability of our method in discriminating drug combinations.

To provide quantitative results, we applied the novelty detection algorithm to drug combination data (Table 3). All the drug combination groups were detected as novel. These results were confirmed in the LDA projection plot (FIG. 6b), where the drug combination groups form distinct clusters and separate from the reference set. It is interesting to find that when the concentration of certain single drug treatment increases in drug combinations, its prediction score is also increased and inclined to this single drug treatment. For instance, for Eve-Lap (1-2), whose concentration of lapatinib doubled compared with Eve-Lap (1-1), its mean prediction score is improved to slightly above zero with the closest known drug as lapatinib. The LDA plot also showed its closer distribution to lapatinib (EGFR/HER2 inhibitor). This suggests the possible dose responses in our method when evaluating drug combinations. Overall, our method has the ability to discriminate drug combinations in both qualitative and quantitative manners.

TABLE 3
Prediction of drug combinations as known
or novel using novelty detection:

| | | Outlier | |
|---|---|---|---|
| Drugs | Mean prediction score | percentage | Prediction |
| Eve-Lap (1-1) | −0.5880 | 79.40% | Novel |
| Eve-Lap (1-2) | 0.05565 | 47.22% | Novel |
| Eve-Dox (2-1) | −0.8683 | 93.41% | Novel |
| Eve-Dox (1-2) | −0.1482 | 57.41% | Novel |

Metabolic heterogeneity analysis revealed cell subpopulation during drug treatments: A unique advantage of measuring single-cell drug response versus ensemble response is the ability to assess cell-to-cell heterogeneity, which is known as the major cause of drug resistance [65]. Cellular metabolism has been suggested to be a good readout of cell functional heterogeneity [66]. Thus, we introduced a series of statistical strategies and data visualization methods to analyze cell metabolic heterogeneity during drug treatments.

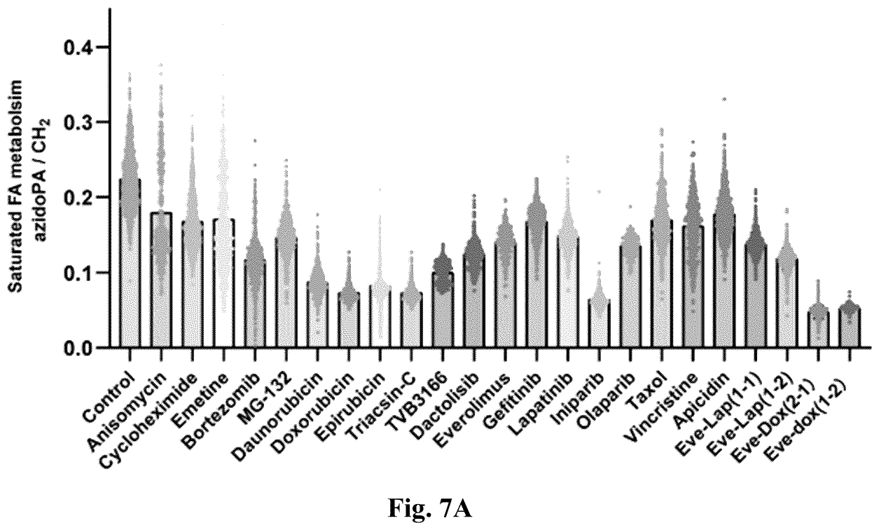

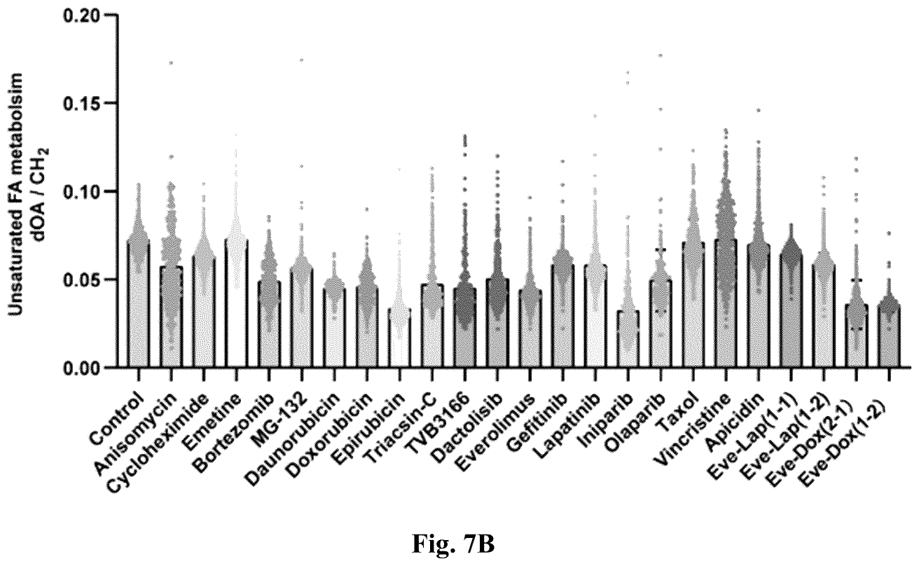

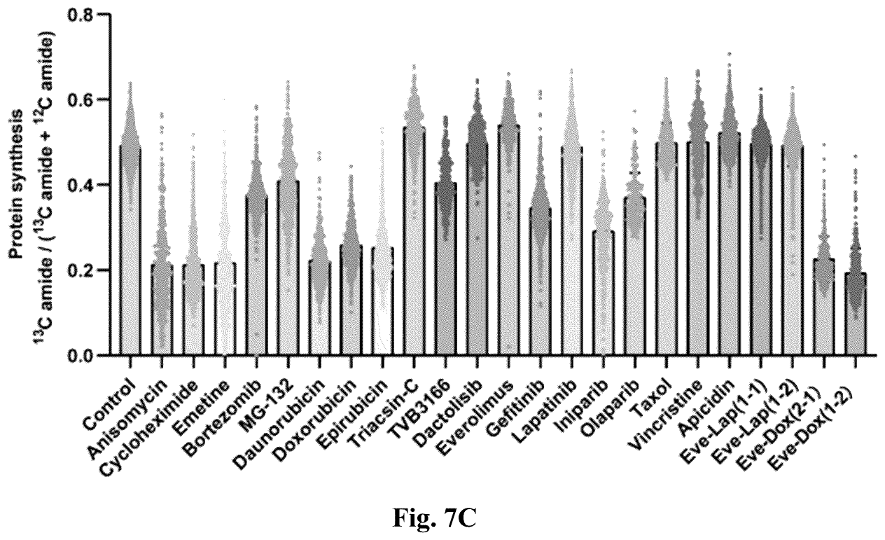

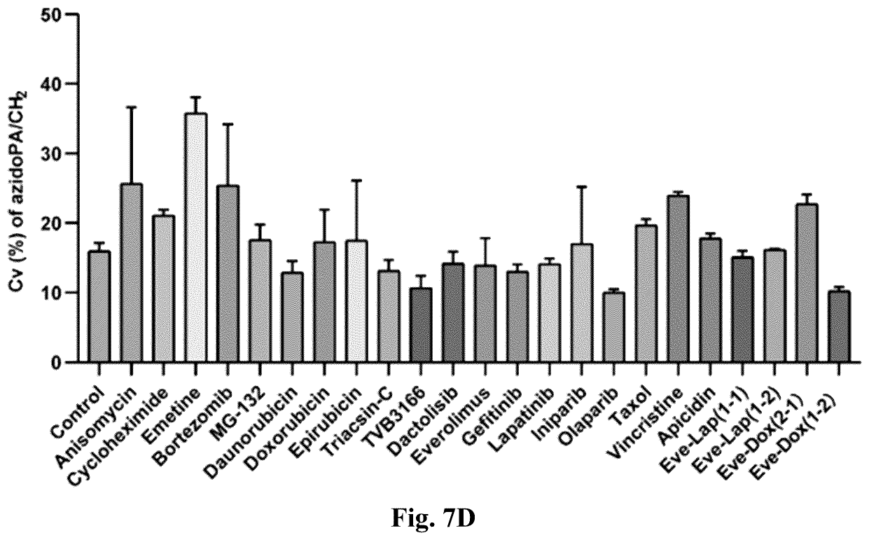

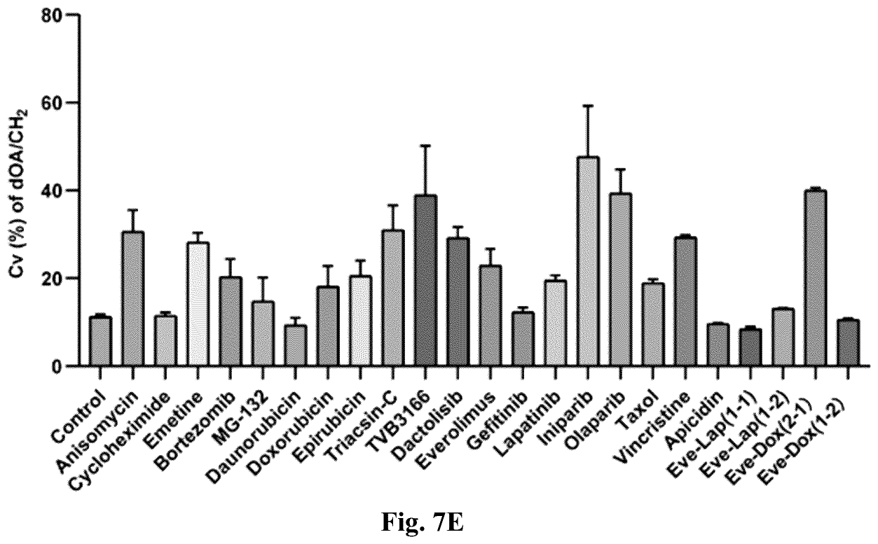

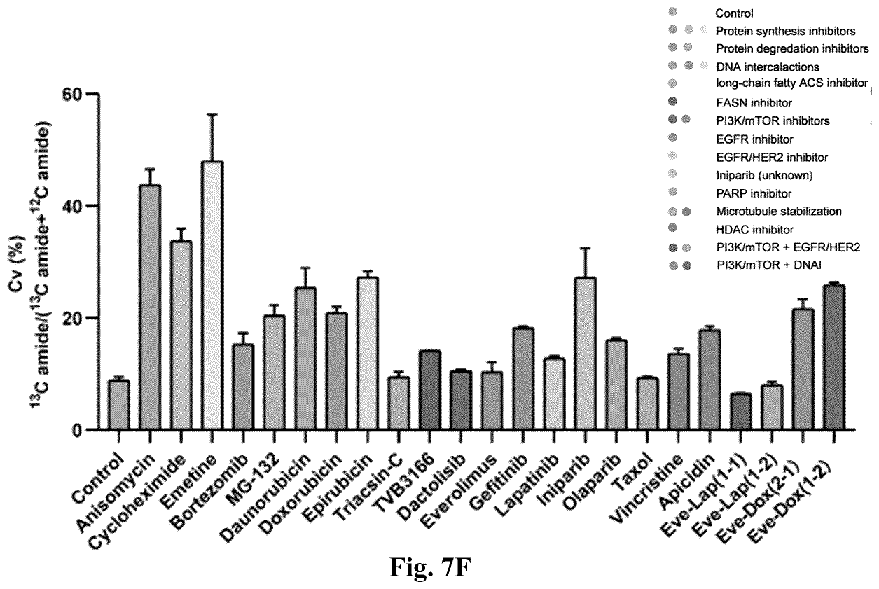

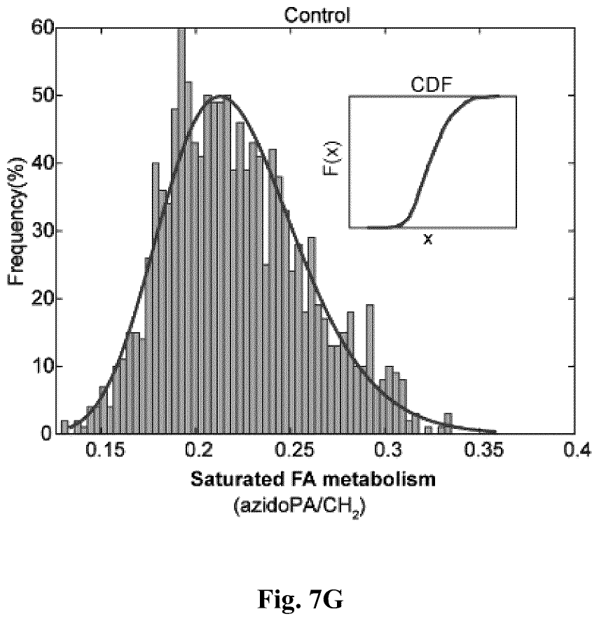

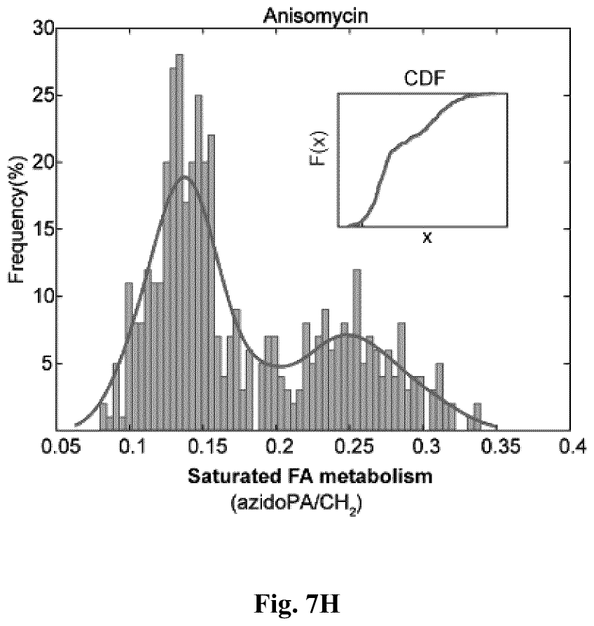

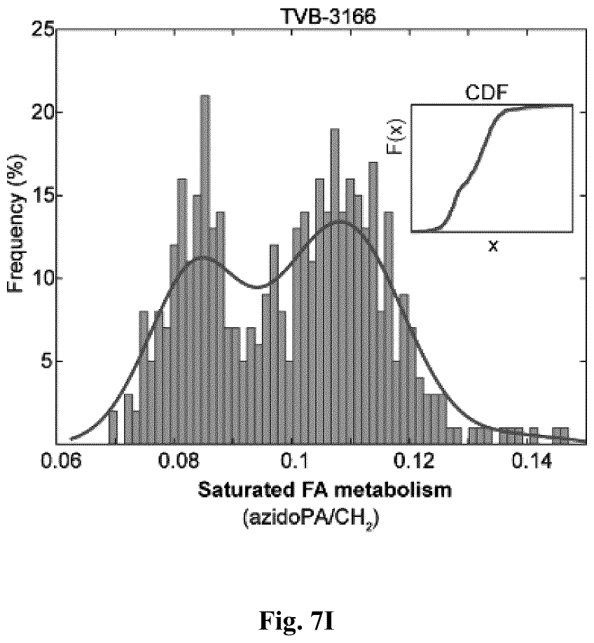

We used composite graphs integrating individual values and box plots to map the distributions of single metabolic activities of all the tested drug treatments (23 drug treatments plus control, FIG. 7a). These graphs further confirmed our findings that drugs belonging to the same MoA, which are encoded by similar colors (e.g. anisomycin, cycloheximide and emetine for protein synthesis), induce similar metabolic responses. Moreover, single-cell data with far more active metabolic activities than average can be directly visualized in the plots. These cells showed high survivability with vigorous metabolism even under drug treatment, which might be relevant to drug resistance.

We then calculated the Cv values of the three metabolic activities, respectively, to quantify metabolic heterogeneity (FIG. 3b). Interestingly, the control group possesses almost minimal Cv values in all three metabolic activities, whereas cells treated with single drugs showed elevated Cv values in general. Several drug combination groups (e.g. Eve-Lap), however, have similar or even lower Cv values compared with control. This suggests decreased cell heterogeneity when using certain drug combinations. Considering that the addition of everolimus improves the sensitivity and reduces the resistance of lapatinib [67], our result might be relevant to the reduced drug resistance in this drug combination group. Together, these results showed the ability of our method to quantify and evaluate cell heterogeneity under different drug treatment conditions.

To find out whether subpopulation (i.e., distinct phenotypic states) exists among cancer cells under different drug treatments, we introduced Hartigan's dip test, which infers the unimodality/multimodality of distributions by detecting the occurred dip in the cumulative distribution function (CDF) [68]. We applied the dip test on all three metabolic activities from 23 drug treatments plus the control (72 distributions in total). Interestingly, we discovered multimodal distributions of saturated FA metabolism under two different drug treatments (with p-value <0.05). As shown in FIG. 7c, cells treated with either anisomycin (protein synthesis inhibitor) or TVB-3166 (FASN inhibitor) showed two clearly resolved peaks (multimodal) whereas the control group is apparently single-peaked (unimodal). These distributions were further confirmed in other batches. Such findings prove the necessity of single-cell drug response measurement, as the conventional bulk measurement would average over the entire population and miss the existence of any underlying sub-populations. If our technique were not sufficiently sensitive to different phenotypic states or if the technique did not have sufficient throughput to measure enough number of cells, this result would not be possible.

### Discussion

In this work, we developed “Chemical Painting”, a new method integrating MIR imaging, multiplexed vibrational probes and enhanced data analysis pipeline, for measuring single-cell drug responses. Our method showed high sensitivity in discriminating cell phenotypes perturbed by a broad range of drugs. Utilizing this property, we built a robust machine learning classifier with high accuracy and minimal batch effects to predict drug MoAs at single-cell level. We further designed an algorithm to identify drugs with novel MoAs and assess drug combinations. These studies serve as a solid foundation to further apply our method toward phenotypic drug discovery. Moreover, through metabolic heterogeneity analysis, we identified cell sub-populations (distinct phenotypes) after drug treatment, which might be useful for accessing drug resistance.

Chemical Painting offers a novel and alternative way for cell phenotyping after drug perturbation. Different from conventional image-based profiling methods (e.g. Cell Painting) measuring the fluorescence signals from organelle staining, our method measures the biochemical and metabolic compositions of cells from corresponding vibrational spectra. Given the consensus that metabolism provides the readout closest to cell phenotypes [69,70], the features provided in our method might be more relevant to cell states. In addition, our method is intrinsically high-content with hundreds of features (vibrational peaks). Extra feature extraction algorithms are not required as in Cell Painting [8], which might generate redundant features and cause additional downstream analysis labor. Another advantage of our method is its minimal batch effects, which mitigates the essential concern of phenotypic assays in capturing batch information over biological information [9]. This further reduces the complexity of experimental design and data analysis to correct batch effects, which are still in the research phase [71].

The current throughput can be further improved by using DFIR imaging, which has been reported to be 10-100 times faster than FTIR imaging [56,72-74]. As we have tested that around 50 features are required to achieve accurate drug MOA prediction (FIG. 4f), this marvelous speed can be availed of by DFIR imaging with selective frequency imaging.

Besides the demonstrated applications, our method can also be applied in multiple other areas. For drug discovery, it can be used for lead generation after drug screening to select candidates that have been narrowed down [3]. The measured single-cell drug response from our method should provide valuable insights into drug MoAs and drug resistance of the hits. In terms of precision medicine, patient-derived organoids (PDO) have emerged as robust preclinical models for evaluating drug responses and improving drug treatment [77]. Although FTIR imaging does not have 3D sectioning ability, photothermal MIR imaging [78] can potentially perform volumetric imaging for PDO model. Additionally, as a profiling method, Chemical Painting can be applied to detect cell phenotypes perturbed by other genetic strategies, such as RNA interference [80] and CRISPR screening [81], to provide insights into genetic interactions. Overall, as an biochemical profiling method, Chemical Painting can be further tailored for applications in diverse areas.

A previous study (Flower, K. R. et al. Synchrotron FTIR analysis of drug treated ovarian A2780 cells: an ability to differentiate cell response to different drugs? Analyst 136, 498-507 (2011)) showed label-free mid-infrared imaging/spectroscopy failed to discriminate the mid-infrared spectra of several drug-treated cells from control at the single-cell level. Thus, before the present disclosure, there was no notion that mid-infrared imaging or spectroscopy could be used in a way capable of classifying drug mechanism of action (MoA) nor phenotypic drug discovery, with or without probes.

In addition, it was unknown whether multiple vibrational probes could be used coordinately together instead of working against each other in, e.g., human cancer cells. Moreover, it was unknown whether their signals can be captured simultaneously and also be distinguishable by mid-infrared imaging/spectroscopy.

Furthermore, earlier work focused on the metabolic imaging of biological samples targeting single vibrational peaks. In embodiments the present methods can employ all features from both multiplexed vibrational probes and label-free regions to form a biochemical profile. This high-content biochemical profile integrating all the features shows sufficiently high sensitivity to classify drug mechanisms. In this regard, it was determined in the present disclosure that when focusing on single vibrational peaks/ratios only from the vibrational probes, the prediction accuracy of 10 categories of drug MoAs drops to 39.47% for 13C-AA only, 37.03% for azido-PA only and 32.61% for d₃₄-OA. If uses all three single vibrational peaks, the prediction accuracy increases to 69.52%, but still dramatically lower than the 99.81% accuracy achieved by using the novel approach combining all the features. (The predictions were based on data from the same batch).

Cell Painting papers (see, e.g., Bray, M.-A. et al. Cell Painting, a high-content image-based assay for morphological profiling using multiplexed fluorescent dyes. Nat. Protoc. 11, 1757-1774 (2016)) emphasize the importance of using general fluorescence stains such as cell organelle stains to detect a broad range of phenotypic effects upon compound treatments. It states “The choice of stains for the Cell Painting protocol was based on the desire to detect a broad range of phenotypic effects upon compound treatment or genetic perturbation, while keeping the assay inexpensive and straightforward to implement using conventional sample preparation and imaging equipment.” In the present disclosure, the label-free vibrational peaks originating from biological macromolecules in cells can be seen as general features, while the features from introduced vibrational peaks targeting specific metabolism can be seen as specific features. Before the present disclosure, it was unknown whether combining general features from label-free regions and specific features from vibrational peaks could be used, or is indeed helpful, in discriminating drug MoAs. In fact, it was certainly possible that those specific features could interfere with the general features from label-free regions and further lower its ability to differentiate/classify drug MoAs. For instance, the 13C-AA probe utilized in the present has its vibrational peak at around 1616 cm-1, which overlapped with the label-free amide peak from proteins in cells centered at 1650 cm-1. As the amide peak is quite sensitive to protein secondary structures (Kong, Jilie, and Shaoning Yu. “Fourier transform infrared spectroscopic analysis of protein secondary structures.” Acta biochimica et biophysica Sinica 39.8 (2007): 549-559), it is possible that the introduced 13C-AA could lower the sensitivity of the amide peak to protein secondary structures and further decrease the sensitivity of infrared spectrum in discriminating cell spectra treated by different drugs. Only after doing empirical studies and testing was it determined that combining all the features together is useful for classifying drug MoAs and phenotypic drug discovery.

### Methods

Cell line and materials. MDA-MB-231 (ATCC HTB-26) was purchased from ATCC. For reagents, azido-palmitic acid (1346) was purchased from Click chemistry tools; algal amino acid mixture (U-13C, 97-99%, CLM-1548) was purchased from Cambridge; deuterated oleic acid (683582) was purchased from Sigma-Aldrich. For drugs, anisomycin (A9789), bortezomib (179324-69-7), cycloheximide (01810), emetine (SMB01061), everolimus (94687), epirubicin hydrochloride (E9406), gefitinib (184475-35-2), lapatinib (231277-92-2), TVB-3166 (SML1694) were purchased from Sigma-Aldrich. Daunorubicin hydrochloride (AAJ60224MA), iniparib (AC469161000), triacsin C (24-721-00U), doxorubicin hydrochloride (BP25165), MG 132 (AAJ63250LB0), dactolisib (NC0298104) were purchased from Fisher Scientific. For cell culture agents, DMEM medium (11965), FBS (10082), penicillin/streptomycin (1514), were purchased from ThermoFisher Scientific. CaF₂ substrates (CAFP13-1) were purchased from Crystran.

Probe preparation and media recipe for cell labeling. Azido palmitic acid-bovine serum albumin (BSA) solution. For the solution, couple azido-palmitic acid with BSA to prepare a 2-mM stock solution. Prepare 20 mM sodium palmitic acid solution by dissolving palmitic acid in NaOH solution with the following recipe: azido-PA (5.5 mg)+1.0 ml dd-H₂O+35 ul 1M NaOH. Mix and incubate the solution in 70° C. water baths until no oil droplets are visible. Then slowly add the sodium palmitic acid solution into 2.7 ml 20% BSA under room temperature water baths. Quickly add 6.3 ml DMEM culture medium and filter the solution with a 0.22-?m sterile filter.

¹³C-amino acids DMEM: 4 mg ml/1 algae ¹³C-amino acids mix was dissolved in dd-H2O with 10% FBS and 1% penicillin.

Deuterated oleic acid-bovine serum albumin (BSA) solution: For the solution, couple d34-oleic acid with BSA to prepare a 2-mM stock solution. Prepare 20 mM oleic acid solution by dissolving oleic acid in NaOH solution with the following recipe: d₃₄ oleic acid (6.3 mg)+1.0 ml dd-H₂O+24 ul 1 M NaOH. Mix and incubate the solution in 70° C. water baths until no oil droplets are visible. Then slowly add the d₃₄ oleic acid solution into 2.7 ml 20% BSA under room temperature water baths. Quickly add 6.3 ml DMEM culture medium and filter the solution with a 0.22-um sterile filter.

Cell culture. MDA-MB-231 cells were cultured in DMEM media supplemented with 10% FBS and 1% penicillin. Cells were grown in a humidified atmosphere containing 5% CO₂ at 37° C. in the incubator. At ˜80% confluence, cells were dissociated with trypsin and passaged.

Cell viability assay and drug IC50 calculus. The IC50 values of the drugs were determined by Alamar blue assay. Cells were seeded at 10,000 per well in 96-well plates. After 24 h, the cells were washed twice with phosphate buffered saline (PBS) and treated with drugs at different concentrations in cell culture media for 48 hrs. Each drug concentration has 6-8 replicates. After the drug treatments, cells were washed with PBS twice and the cell viability was determined by Alamar blue assay following the manufacturer's protocol (Invitrogen) using plate reader. The IC50 values were determined by fitting the data using a dose response model with variable Hill slope built in Prism.

Drug combination and synergy score calculus. Cell viability assay was performed on cells treated by drug combinations to evaluate synergy scores. Everolimus-Lapatinib and Everolimus-Doxorubicin were chosen as model systems to study drug combinations. Each drug concentration combination has 5 replicates. To calculate synergy scores, an open-source package SynergyFinder64 was applied. The synergy scores were calculated based on the ZIP model available in the package.

Sample preparation for drug-treated cells with labeling. MDA-MB-231 cells were seeded on clean CaF₂ substrates with 5×10⁴ cells per well in cell culture media (DMEM, 10% FBS, 1% penicillin) overnight. Then the culture media was replaced by ¹³C-amino acids DMEM with 50 μM azido-palmitic acid, 50 μM d₃₄ oleic acid, either single drug at its IC50 concentration or drug combinations at chosen concentrations. Drugs were prepared in 100% DMSO and diluted to 0.1% DMSO in labeling media. For the control group, only cell labeling media with 0.1% DMSO was added (without any drugs). Cells were treated for 48 hrs. After that, cells were fixed by 4% PFA at room temperature for 15 min and washed three times with PBS buffer and five times with dd-H₂O. The samples were then air-dried before imaging.

FTIR imaging. Agilent Cary 620 Imaging FTIR equipped with an Agilent 670-IR spectrometer and 128×128-pixels FPA mercury cadmium telluride (MCT) detector was used in the transmission mode. A background spectrum was collected on a clean CaF₂ substrate using 128 scans at 8 cm-1 spectral resolution. Cell spectra were recorded using 64-128 scans at 8 cm-1 spectral resolution. A ×25 IR objective (pixel size, 3.3 um, 0.81 numerical aperture (NA)) was used for cell imaging.

Data preprocessing. Data preprocessing was performed using both the commercial software Cytospec and home-built MATLAB scripts with the following steps: 1) PCA noise reduction to denoise the spectra; 2) quality test to remove pixels with low SNR in both the fingerprint region and the cell silent region; 3) rubber-band baseline correction for spectral correction; 4) min-max data normalization; 5) single-cell segmentation on cell images using Cell Profiler. 6). single-cell spectrum extraction applying generated single-cell masks on the processed FTIR imaging data.

Linear unmixing. To obtain the pure signal of the three vibrational probes, linear unmixing was performed. For ¹³C-amide I and ¹²C-amide I, the unmixing coefficients were measured from spectra of bacteria growing in the media with ¹²C6-glucose or ¹³C6-glucose as the only carbon source [43]. The final linear combination coefficients are:

$${}^{12} C - amide = 1.2813 * A 1651 - 0.5484 * A 1616$$

$${}^{13} C - amide = 1.2813 * A 1616 - 0.6573 * A 1651$$

For azido-PA (2096 cm-1) and d₃₄OA (2092 cm-1 and 2196 cm-1), the unmixing coefficients were measured from spectra of cells growing in media added with either azido-PA or d₃₄OA. The final linear combination coefficients are:

$${d}_{34} O A = 1.0379 * A 2196 - 0.052 * A 2096$$

$$azido - P A = 1.0379 * A 2096 - 0.7559 * A 2196$$

3D scatter plots. To construct the 3D scatter graph, three ratio values (azido-PA/CH₂, d₃₄OA/CH₂, ¹³C amide I/(¹³C amide I+¹²C amide I)) were calculated for each cell based on thee extracted single-cell spectrum data. These paired ratio values of each cell were then plotted on the 3D graph using MATLAB with shaded areas indicating error ellipse with 80% confidence. Color coding was used to separate the data points from different drug treatments. Cells treated by drugs with the same MoA have similar color coding.

Machine learning. The tests of all the machine learning classifiers were performed using Python (Scikit-learn). Specifically, single-cell data was split into 70% training and 30% testing with labels corresponding to drug MoAs. The hyper-parameters of each classifier were carefully tuned using RandomizedSearchCv in Scikit-learn. The performances of these classifiers were evaluated in the testing dataset using accuracy as the main metric. To test the performance of classifiers on batch effects, single-cell data collected from different batches were loaded as another testing dataset. For feature importances of LDA classifier, permutation feature importance (Scikit-learn) was used. The intrinsic feature importance values from random forest classifier were also extracted.

Novelty detection algorithm. In the first step, the Mahalanobis distances between the test compound and each reference class in the annotated dataset were calculated by home-built codes in python. Drug class with minimum distance was selected. Next, isolation forest, which is available in Python, was imported to predict whether single-cell data from the test compound is known (inliers) or novel (outliers) to the selected drug class. The mean of the prediction results from single-cell data is reported as mean prediction score.

Metabolic heterogeneity analysis. Composite graphs integrating individual values and box plots of three metabolic activities were built by Prism. Cv values were calculated using Matlab by randomly splitting single-cell data of each drug treatment into three groups. For Hartigan's dip tests, the distribution of each metabolic activities from all groups (15 drugs+control) were tested using diptest package available in Python. The histograms were plotted with kernel density fitting.

UMAP. UMAP graphs were plotted based on single-cell spectrum data using available packages in Python. Either data of 3 metabolic ratios or full-spectrum data were tested. The UMAP plot of drug combinations utilized full-spectrum data. Data were z-score normalized as input for UMAP.

Statistical analysis. Hartigan's dip test was performed using diptest package available in Python. The p-values of saturated FA metabolism (azido-PA/CH₂) for anisomycin and TVB-3166 are 0.047, 8.40×10-5, respectively. In FIG. 2, for control group, the cell number is 1000; for anisomycin, the cell number is 502; for cycloheximide, the cell number is 700; for emetine, the cell number is 526; for bortezomib, the cell number is 583; for MG-132, the cell number is 579; for daunorubicin, the cell number is 512; for doxorubicin, the cell number is 509; for epirubicin, the cell number is 950; for triacsin-C, the cell number is 534; for TVB3166, the cell number is 503; for dactolisib, the cell number is 513; for everolimus, the cell number is 495; for gefitinib, the cell number is 821; for lapatinib, the cell number is 951; for iniparib, the cell number is 950; for epirubicin, the cell number is 487. The total cell number is 11,115 for all 16 groups (with control). FIG. 4 shares the same statistics as FIG. 2. In FIG. 3, to ensure class balances, the single-cell data number from each drug MoA is confined to be around 500-700, making up a dataset with cell number of 6298 in total. In FIG. 5, for control group, the cell number is 609; for everolimus group, the cell number is 740; for lapatinib group, the cell number is 873; for doxorubicin group, the cell number is 485; for Eve-Lap (1-1) group, the cell number is 988; for Eve-Lap (1-2) group, the cell number is 976; for Eve-Dox (2-1) group, the cell number is 471; for Eve-Dox (1-2) group, the cell number is 459. The total cell number is 5,601. For experiment replicates, experiments in FIG. 2 have been repeated in 3 different batches with similar results. The Cv values in FIG. 4 are calculated based on n=3 cell cultures. The Cv values in FIG. 5 are calculated based on n=3 cell cultures.

### REFERENCES

1. Kaddurah-Daouk, R., Kristal, B. S. & Weinshilboum, R. M. Metabolomics: a global biochemical approach to drug response and disease. Annu. Rev. Pharmacol. Toxicol. 48, 653-683 (2008).
2. Wishart, D. S. Emerging applications of metabolomics in drug discovery and precision medicine. Nat. Rev. Drug Discov. 15, 473-484 (2016).
3. Chandrasekaran, S. N., Ceulemans, H., Boyd, J. D. & Carpenter, A. E. Image-based profiling for drug discovery: due for a machine-learning upgrade? Nat. Rev. Drug Discov. 20, 145-159 (2021).
4. Wang, Y.-Y. et al. CeDR Atlas: a knowledgebase of cellular drug response. Nucleic Acids Res. 50, D1164-D1171 (2022).
5. Zhao, Y. et al. Measurement methods of single cell drug response. Talanta 239, 123035 (2022).
6. Fritzsch, F. S. O., Dusny, C., Frick, O. & Schmid, A. Single-cell analysis in biotechnology, systems biology, and biocatalysis. Annu. Rev. Chem. Biomol. Eng. 3, 129-155 (2012).
7. Walsh, A. J. et al. Quantitative optical imaging of primary tumor organoid metabolism predicts drug response in breast cancer. Cancer Res. 74, 5184-5194 (2014).
8. Bray, M.-A. et al. Cell Painting, a high-content image-based assay for morphological profiling using multiplexed fluorescent dyes. Nat. Protoc. 11, 1757-1774 (2016).
9. Qian, W. W. et al. Batch equalization with a generative adversarial network. Bioinformatics 36, 1875-1883 (2020).
10. Duncan, K. D., Fyrestam, J. & Lanekoff, I. Advances in mass spectrometry based single-cell metabolomics. Analyst 144, 782-793 (2019).
11. Zhang, L. & Vertes, A. Single-cell mass spectrometry approaches to explore cellular heterogeneity. Angew. Chem. Int. Ed Engl. 57, 4466-4477 (2018).
12. Spitzer, M. H. & Nolan, G. P. Mass cytometry: Single cells, many features. Cell 165, 780-791 (2016).
13. Bendall, S. C. et al. Single-cell mass cytometry of differential immune and drug responses across a human hematopoietic continuum. Science 332, 687-696 (2011).
14. Bodenmiller, B. et al. Multiplexed mass cytometry profiling of cellular states perturbed by small-molecule regulators. Nat. Biotechnol. 30, 858-867 (2012).
15. Heath, J. R., Ribas, A. & Mischel, P. S. Single-cell analysis tools for drug discovery and development. Nat. Rev. Drug Discov. 15, 204-216 (2016).
16. Cohen, Y. C. et al. Identification of resistance pathways and therapeutic targets in relapsed multiple myeloma patients through single-cell sequencing. Nat. Med. 27, 491-503 (2021).
17. Kim, K.-T. et al. Single-cell mRNA sequencing identifies subclonal heterogeneity in anti-cancer drug responses of lung adenocarcinoma cells. Genome Biol. 16, 127 (2015).
18. Jamieson, L. E. & Byrne, H. J. Vibrational spectroscopy as a tool for studying drug-cell interaction: Could high throughput vibrational spectroscopic screening improve drug development? Vib. Spectrosc. 91, 16-30 (2017).
19. Butler, H. J. et al. Using Raman spectroscopy to characterize biological materials. Nat. Protoc. 11, 664-687 (2016).
20. Baker, M. J. et al. Using Fourier transform IR spectroscopy to analyze biological materials. Nat. Protoc. 9, 1771-1791 (2014).
21. Beljebbar, A., Sockalingum, G. D., Morjani, H. & Manfait, M. Raman and SERS microspectroscopy on living cells: a promising tool toward cellular drug response and medical diagnosis. in Biomedical Applications of Raman Spectroscopy (ed. Morris, M. D.) (SPIE, 1999). doi: 10.1117/12.345399.
22. Zhao, Z., Chen, C., Xiong, H., Ji, J. & Min, W. Metabolic activity phenotyping of single cells with multiplexed vibrational probes. Anal. Chem. 92, 9603-9612 (2020).
23. Buckmaster, R., Asphahani, F., Thein, M., Xu, J. & Zhang, M. Detection of drug-induced cellular changes using confocal Raman spectroscopy on patterned single-cell biosensors. Analyst 134, 1440-1446 (2009).
24. Hekmatara, M., Heidari Baladehi, M., Ji, Y. & Xu, J. D2O-probed Raman microspectroscopy distinguishes the metabolic dynamics of macromolecules in organellar anticancer drug response. Anal. Chem. 93, 2125-2134 (2021).
25. Wang, H. et al. Confocal Raman microspectral analysis and imaging of the drug response of osteosarcoma to cisplatin. Anal. Methods 13, 2527-2536 (2021).
26. Zhang, Y. et al. Dynamic characterization of drug resistance and heterogeneity of the gastric cancer cell BGC823 using single-cell Raman spectroscopy. Analyst 143, 164-174 (2017).
27. Farhane, Z., Nawaz, H., Bonnier, F. & Byrne, H. J. In vitro label-free screening of chemotherapeutic drugs using Raman microspectroscopy: Towards a new paradigm of spectralomics. J. Biophotonics 11, (2018).
28. Tipping, W. J., Lee, M., Serrels, A., Brunton, V. G. & Hulme, A. N. Stimulated Raman scattering microscopy: an emerging tool for drug discovery. Chem. Soc. Rev. 45, 2075-2089 (2016).
29. Tipping, W. J., Lee, M., Serrels, A., Brunton, V. G. & Hulme, A. N. Imaging drug uptake by bioorthogonal stimulated Raman scattering microscopy. Chem. Sci. 8, 5606-5615 (2017).
30. Tipping, W. J. et al. Stimulated Raman scattering microscopy with spectral phasor analysis: applications in assessing drug-cell interactions. Chem. Sci. 13, 3468-3476 (2022).
31. Tipping, W. J. et al. Temporal imaging of drug dynamics in live cells using stimulated Raman scattering microscopy and a perfusion cell culture system. RSC Chem. Biol. 3, 1154-1164 (2022).
32. Ma, J., Pazos, I. M., Zhang, W., Culik, R. M. & Gai, F. Site-specific infrared probes of proteins. Annu. Rev. Phys. Chem. 66, 357-377 (2015).
33. Draux, F. et al. IR spectroscopy reveals effect of non-cytotoxic doses of anti-tumour drug on cancer cells. Anal. Bioanal. Chem. 395, 2293-2301 (2009).
34. Derenne, A., Gasper, R. & Goormaghtigh, E. The FTIR spectrum of prostate cancer cells allows the classification of anticancer drugs according to their mode of action. Analyst 136, 1134-1141 (2011).
35. Derenne, A., Verdonck, M. & Goormaghtigh, E. The effect of anticancer drugs on seven cell lines monitored by FTIR spectroscopy. Analyst 137, 3255-3264 (2012).
36. Gasper, R., Dewelle, J., Kiss, R., Mijatovic, T. & Goormaghtigh, E. IR spectroscopy as a new tool for evidencing antitumor drug signatures. Biochim. Biophys. Acta 1788, 1263-1270 (2009).
37. Mignolet, A. & Goormaghtigh, E. High throughput absorbance spectra of cancerous cells: a microscopic investigation of spectral artifacts. Analyst 140, 2393-2401 (2015).
38. Mignolet, A., Mathieu, V. & Goormaghtigh, E. HTS-FTIR spectroscopy allows the classification of polyphenols according to their differential effects on the MDA-MB-231 breast cancer cell line. Analyst 142, 1244-1257 (2017).
39. Mignolet, A., Derenne, A., Smolina, M., Wood, B. R. & Goormaghtigh, E. FTIR spectral signature of anticancer drugs. Can drug mode of action be identified? Biochim. Biophys. Acta 1864, 85-101 (2016).
40. Altharawi, A., Rahman, K. M. & Chan, K. L. A. Towards identifying the mode of action of drugs using live-cell FTIR spectroscopy. Analyst 144, 2725-2735 (2019).
41. Flower, K. R. et al. Synchrotron FTIR analysis of drug treated ovarian A2780 cells: an ability to differentiate cell response to different drugs? Analyst 136, 498-507 (2011).
42. Denbigh, J. L. et al. Probing the action of a novel anti-leukaemic drug therapy at the single cell level using modern vibrational spectroscopy techniques. Sci. Rep. 7, 2649 (2017).
43. Shi, L. et al. Mid-infrared metabolic imaging with vibrational probes. Nat. Methods 17, 844-851 (2020).
44. Liu, X. et al. Towards mapping mouse metabolic tissue atlas by mid-infrared imaging with heavy water labeling. Adv. Sci. 9, e2105437 (2022).
45. Johannessen, C. M., Clemons, P. A. & Wagner, B. K. Integrating phenotypic small-molecule profiling and human genetics: the next phase in drug discovery. Trends Genet. 31, 16-23 (2015).
46. Büttner, F. H. Cell-based assays for high-throughput screening. Expert Opin. Drug Discov. 1, 373-378 (2006).
47. Blay, V., Tolani, B., Ho, S. P. & Arkin, M. R. High-Throughput Screening: today's biochemical and cell-based approaches. Drug Discov. Today 25, 1807-1821 (2020).
48. Stirling, D. R. et al. CellProfiler 4: improvements in speed, utility and usability. BMC Bioinformatics 22, 433 (2021).
49. Pang, B. et al. Drug-induced histone eviction from open chromatin contributes to the chemotherapeutic effects of doxorubicin. Nat. Commun. 4, 1908 (2013).
50. Taymaz-Nikerel, H., Karabekmez, M. E., Eraslan, S. & Kirdar, B. Doxorubicin induces an extensive transcriptional and metabolic rewiring in yeast cells. Sci. Rep. 8, 13672 (2018).
51. Jamdade, V. S. et al. Therapeutic targets of triple-negative breast cancer: a review. Br. J. Pharmacol. 172, 4228-4237 (2015).
52. Kummar, S. et al. Advances in using PARP inhibitors to treat cancer. BMC Med. 10, 25 (2012).
53. Caulfield, S. E., Davis, C. C. & Byers, K. F. Olaparib: A novel therapy for metastatic breast cancer in patients with a BRCA1/2 mutation. J. Adv. Pract. Oncol. 10, 167-174 (2019).
54. McInnes, L., Healy, J. & Melville, J. UMAP: Uniform Manifold Approximation and Projection for Dimension Reduction. arXiv [stat.ML] (2018).
55. Moffat, J. G., Rudolph, J. & Bailey, D. Phenotypic screening in cancer drug discovery—past, present and future. Nat. Rev. Drug Discov. 13, 588-602 (2014).
56. Yeh, K., Kenkel, S., Liu, J.-N. & Bhargava, R. Fast infrared chemical imaging with a quantum cascade laser. Anal. Chem. 87, 485-493 (2015).
57. Rana, S. et al. A multichannel nanosensor for instantaneous readout of cancer drug mechanisms. Nat. Nanotechnol. 10, 65-69 (2015).
58. Vincent, F. et al. Phenotypic drug discovery: recent successes, lessons learned and new directions. Nat. Rev. Drug Discov. 21, 899-914 (2022).
59. Liu, F. T., Ting, K. M. & Zhou, Z.-H. Isolation Forest. in 2008 Eighth IEEE International Conference on Data Mining (IEEE, 2008). doi: 10.1109/icdm.2008.17.
60. Liang, H. & Tan, A. R. Iniparib, a PARP1 inhibitor for the potential treatment of cancer, including triple-negative breast cancer. IDrugs 13, 646-656 (2010).
61. Mateo, J., Ong, M., Tan, D. S. P., Gonzalez, M. A. & de Bono, J. S. Appraising iniparib, the PARP inhibitor that never was—what must we learn? Nat. Rev. Clin. Oncol. 10, 688-696 (2013).
62. Bayat Mokhtari, R. et al. Combination therapy in combating cancer. Oncotarget 8, 38022-38043 (2017).
63. Chalakur-Ramireddy, N. K. R. & Pakala, S. B. Combined drug therapeutic strategies for the effective treatment of Triple Negative Breast Cancer. Biosci. Rep. 38, BSR20171357 (2018).
64. Ianevski, A., Giri, A. K. & Aittokallio, T. SynergyFinder 2.0: visual analytics of multi-drug combination synergies. Nucleic Acids Res. 48, W488-W493 (2020).
65. Wang, R., Jin, C. & Hu, X. Evidence of drug-response heterogeneity rapidly generated from a single cancer cell. Oncotarget 8, 41113-41124 (2017).
66. Kim, J. & DeBerardinis, R. J. Mechanisms and implications of metabolic heterogeneity in cancer. Cell Metab. 30, 434-446 (2019).
67. Gayle, S. S., Arnold, S. L. M., O'Regan, R. M. & Nahta, R. Pharmacologic inhibition of mTOR improves lapatinib sensitivity in HER2-overexpressing breast cancer cells with primary trastuzumab resistance. Anticancer Agents Med. Chem. 12, 151-162 (2012).
68. Hartigan, J. A. & Hartigan, P. M. The dip test of unimodality. Ann. Stat. 13, 70-84 (1985).
69. Seydel, C. Single-cell metabolomics hits its stride. Nat. Methods 18, 1452-1456 (2021).
70. Horgan, R. P. & Kenny, L. C. ‘Omic’ technologies: genomics, transcriptomics, proteomics and metabolomics. The Obstetrician & Gynaecologist vol. 13 189-195 Preprint at https://doi.org/10.1576/toag.13.3.189.27672 (2011).
71. Ando, D. M., McLean, C. Y. & Berndl, M. Improving phenotypic measurements in high-content imaging screens. bioRxiv (2017) doi: 10.1101/161422.
72. Bird, B. & Baker, M. J. Quantum cascade lasers in biomedical infrared imaging. Trends Biotechnol. 33, 557-558 (2015).
73. Ogunleke, A. et al. Fourier-transform vs. quantum-cascade-laser infrared microscopes for histo-pathology: From lab to hospital? Trends Analyt. Chem. 89, 190-196 (2017).
74. Bassan, P., Weida, M. J., Rowlette, J. & Gardner, P. Large scale infrared imaging of tissue micro arrays (TMAs) using a tunable Quantum Cascade Laser (QCL) based microscope. Analyst 139, 3856-3859 (2014).
75. Hofmarcher, M., Rumetshofer, E., Clevert, D.-A., Hochreiter, S. & Klambauer, G. Accurate prediction of biological assays with high-throughput microscopy images and convolutional networks. J. Chem. Inf. Model. 59, 1163-1171 (2019).
76. Zhang, L., Zhang, Y. D., Zhao, P. & Huang, S.-M. Predicting drug-drug interactions: an FDA perspective. AAPS J. 11, 300-306 (2009).
77. Vlachogiannis, G. et al. Patient-derived organoids model treatment response of metastatic gastrointestinal cancers. Science 359, 920-926 (2018).
78. Zhang, Y. et al. Single-shot Volumetric Chemical Imaging by Mid-Infrared Photothermal Fourier Light Field Microscopy. bioRxiv (2022) doi: 10.1101/2022.06.24.497516.
79. Shi, J. et al. High-resolution, high-contrast mid-infrared imaging of fresh biological samples with ultraviolet-localized photoacoustic microscopy. Nat. Photonics 13, 609-615 (2019).
80. Hannon, G. J. RNA interference. Nature 418, 244-251 (2002).
81. Chen, S. et al. Genome-wide CRISPR screen in a mouse model of tumor growth and metastasis. Cell 160, 1246-1260 (2015).

What is claimed:

1. A method of profiling a phenotype of a cell comprising:
introducing a plurality of different infrared (IR) vibrational probes or Raman vibrational probes into the cell, wherein the plurality of different vibrational probes comprise at least two different probes which each emit a quantifiable signal corresponding to a unique cellular parameter; and
obtaining and/or quantifying, by vibrational probe spectroscopy or by vibrational probe imaging, a signal of each vibrational probe so as to determine and/or quantify presence, abundance, and/or spatial location of at least two unique cellular parameters in the cell, so as to thereby profile the phenotype of the cell.

2. The method of claim 1, further comprising obtaining and/or quantifying, by spectroscopy or by imaging one or more quantifiable label-free Raman or IR vibrational signals from the cell which (i) corresponds to one or more cellular parameters and (ii) does not correspond to a vibrational probe, preferably wherein the one or more quantifiable label-free Raman or IR vibrational signals from the cell are intrinsic label-free vibration signals from a biological macromolecule within the cell.

3. The method of claim 1, wherein the cellular parameter comprises a cell metabolism parameter.

4. The method of claim 1, further comprising perturbating the cell before or after profiling the phenotype of the cell.

5. The method claim 1, further comprising administering to the cell, from which the phenotype is to be determined, a biological therapy or a genetic therapy, one or more drugs, candidate drugs, and/or a combination of drug treatments, prior to obtaining prior to obtaining and/or quantifying said signal(s).

6. The method of claim 1, comprising:
introducing a plurality of different IR vibrational probes or Raman vibrational probes into the cell, wherein the plurality of vibrational probes comprises at least two of the following probes:
a probe corresponding to protein synthesis;
a probe corresponding to lipid metabolism, lipid synthesis, and/or lipid uptake;
a probe corresponding to carbohydrate synthesis; and
a probe corresponding to nucleic acid intercalation;
obtaining and/or quantifying, by vibrational probe spectroscopy or by vibrational probe imaging, a signal of each vibrational probe so as to determine and/or quantify the presence, abundance and/or spatial location of protein synthesis, lipid metabolism, lipid uptake, lipid synthesis, carbohydrate synthesis, and/or nucleic acid intercalation in the cell, so as to thereby profile the phenotype of the cell.

7. The method of claim 4, further comprising
i) obtaining and/or quantifying, by vibrational probe spectroscopy or by vibrational probe imaging, a signal of each vibrational probe so as to determine and/or quantify presence, abundance, and/or spatial location of at least two unique cellular parameters in the cell before and after perturbation of the cell, and
ii) comparing the
(1) determined and/or quantified at least two unique cellular parameters before the perturbation of the cell to
(2) the determined and/or quantified at least two unique cellular parameters after the perturbation of the cell, or
so as to phenotypically profile the response of the cell to the perturbation.

8. The method of claim 6, further comprising
i) comparing the determined and/or quantified cellular parameter, or
ii) comparing the determined and/or quantified presence, abundance and/or spatial location of protein synthesis, lipid metabolism, lipid synthesis or lipid uptake, carbohydrate synthesis, and/or nucleic acid intercalation in the cell
to a predetermined or pre-measured control phenotype profile for each so as to phenotypically profile the cell relative to a control phenotype profile.

9. The method of claim 1, wherein the IR vibrational probes introduced into the cell are excited at mid-infrared wavelengths (1600-2300±200 cm⁻¹).

10. The method of claim 1, wherein the IR vibrational probes or Raman vibrational probes introduced into the cell comprise a probe for detecting unsaturated fatty acid uptake.

11. A method of classifying a candidate drug, or a combination of candidate drugs, as having a class of biological mechanism of action at a single-cell level comprising:
obtaining a quantified single-cell test imaging signal or test spectroscopy signal generated by a plurality of IR vibrational probes and quantified from a cell treated with the candidate drug or combination of candidate drugs, the cell comprising therein a plurality of IR vibrational probes comprising at least two of the following probes: a probe for protein synthesis; a probe for lipid metabolism, lipid synthesis, or lipid uptake; a probe for carbohydrate synthesis; and a probe for nucleic acid intercalation;
providing the quantified single-cell test imaging signal or test spectroscopy signal as an input to a machine learning classifier module utilizing a linear discriminant analysis algorithm and a training set comprising a plurality of quantified single-cell imaging signals or spectroscopy signals labeled as to class of biological mechanism of action and quantified from a plurality of cells comprising the IR vibrational probes wherein the cells have been treated with a drug having a known class of biological mechanism of action, the IR vibrational probes comprising at least two of the following probes: a probe for protein synthesis; a probe for lipid metabolism, lipid synthesis or lipid uptake; a probe for carbohydrate synthesis; and a probe for nucleic acid intercalation, such that the machine learning classifier module determines as an output the class of biological mechanism of action of the candidate drug, or of the combination of candidate drugs; and
transmitting the class of biological mechanism of action of the candidate drug or candidate drug combination from the machine learning classifier module to a user device and/or user display, or
a method of classifying a candidate drug, or a combination of candidate drugs, as having a class of biological mechanism of action at a single-cell level comprising:
obtaining a quantified single-cell test imaging signal or test spectroscopy signal generated by a cell comprising a plurality of IR vibrational probes or Raman vibrational probes therein, wherein the plurality of different vibrational probes comprises at least two different probes which each emit a quantifiable signal and correspond to a different cellular parameter, wherein the cell has been treated with the candidate drug or combination;
providing the quantified single-cell test imaging signal or test spectroscopy signal as an input to a machine learning classifier module utilizing a linear discriminant analysis algorithm and a training set comprising a plurality of quantified single-cell imaging signals or spectroscopy signals labeled as to class of biological mechanism of action and quantified from a plurality of cells comprising IR vibrational probes wherein the cells have been treated with a drug having a known class of biological mechanism of action, wherein the plurality of different vibrational probes comprise at least two different probes which each emit a quantifiable signal and correspond to the different cellular parameters, such that the machine learning classifier module determines as an output the class of biological mechanism of action of the candidate drug, or of the combination of candidate drugs; and
transmitting the class of biological mechanism of action from the machine learning classifier module to a user device and/or user display.

12. A method for identifying a novel biological mechanism of action for an unannotated drug, or for identifying a novel biological mechanism of action of an unannotated drug combination, not previously known to have said biological mechanism of action comprising:
providing a phenotype profile of a cell treated with said unannotated drug, or said unannotated drug combination, which phenotype profile has been obtained by the method of claim 1, comparing the phenotype profile with one or more reference classes in a model obtained by applying linear discriminant analysis as a machine learning classifier to a training set of quantified single-cell signals of a plurality of vibrational probes comprising at least two of the following probes: a probe for protein synthesis; a probe for lipid metabolism, lipid synthesis, or lipid uptake; a probe for carbohydrate synthesis; and a probe for nucleic acid intercalation, so as to provide a prediction model of a biological mechanism of action of a candidate drug, or of combination of candidate drugs, for one or more reference classes comprising protein synthesis mechanism of action; lipid metabolism mechanism of action, lipid synthesis mechanism of action, lipid uptake mechanism of action; carbohydrate synthesis mechanism of action; and/or
nucleic acid intercalation mechanism of action;
determining a Mahalanobis distance between the phenotype profile of a cell treated with said unannotated drug, or said unannotated drug combination, with the one or more reference classes in the model so as to determine the reference class(es) with a smallest Mahalanobis distance from the phenotype profile of the so-treated cell;
providing a database of drugs and/or combination of drugs known to have the biological mechanism of action of said reference class(es) with the smallest distance from the phenotype profile of the so-treated cell; and
applying an isolation forest so as to determine whether the unannotated drug or unannotated drug combination having said biological mechanism of action is novel to said reference class.

13. The method of claim 12, wherein the quantified signals comprise vibrational probe signals and spectral feature signals.

14. The method of claim 12, wherein the training set of quantified single-cell signals comprises signals from 3,000 or more single cell drug responses.

15. The method of claim 12, wherein discrete frequency infrared (DFIR) is employed to obtain quantified single-cell signals.

16. The method of claim 12, further comprising obtaining said phenotype profile of a cell treated with said candidate drug, or said combination of candidate drugs.

17. A device comprising (i) a vibrational infrared (IR) detection device comprising a sample chamber, which vibrational IR detection device comprises a radiation source which irradiates the sample chamber with radiation of mid-infrared wavelengths (1000-3500±200 cm⁻¹) and a detector which detects and quantifies absorption spectra of a plurality of mid-infrared probes corresponding to one or more predetermined metabolic processes by a cell within a sample within said sample chamber so as to obtain spatial and quantitative data about said predetermined metabolic processes by a cell, and wherein said device presents to a user thereof the spatial and/or quantitative data from the plurality of mid-infrared probes, or (ii) a Raman spectroscopy detection device comprising a sample chamber, which Raman spectroscopy detection device comprises one or more radiation source lasers which irradiate the sample chamber and a Raman spectroscopy detector which detects and quantifies the Raman spectra of one or more molecules corresponding to one or more predetermined metabolic processes by a cell within a sample within said sample chamber so as to obtain spatial and quantitative data about said predetermined metabolic processes by a cell, and wherein said device presents to a user thereof the spatial and/or quantitative data from the plurality of mid-infrared probes.

18. The device of claim 17, wherein the vibrational IR detection device comprises a FTIR spectrometer.

19. The device of claim 17, wherein the detector comprises a photon/quantum detector or thermal detector.

20. The device of claim 19, wherein the detector comprises an imaging sensor.
